SEQUENCE LISTING (5 sequences)

SEQ ID NO: 1 - protein, 72 aa, Mus musculus
  Amino acid sequence of mouse G-gamma 12 protein subunit
MSSKTASTNSIAQARRTVQQLRLEASIERIKVSKASADLMSYCEEHARSDPLLMGIPTSENPFKDKKTCIIL

SEQ ID NO: 2 - DNA, 4147 bp, Mus musculus
  cDNA sequence for mouse G-gamma 12 protein subunit, variant 1
ctagaattcagcggccgctgaattctaggcgacgacggcgaagagtgagtgccaaggttcatatgggaaggactttggggtgagcatcttctctatttccagctggcttttctgattttcagaaagaagactcatcaaagatgtccagcaagacggcaagcaccaacagcatagcccaagccaggagaactgtgcagcagctgagattggaagcctccatcgaaagaataaaggtctcaaaagcatcagcagacctgatgtcatactgtgaggagcatgcccggagcgaccccctgctgatgggcataccgacctcagaaaacccgttcaaggataagaagacctgcatcatcttatagtggaccaggaagcgccccttgcctcttaacgcaaaccacagcagcaacctgaagggattccttcagcttacctggtaaccacagctagtaactaaaacacccttctctcggaataatagaccctgaagtctctctttttcaagttgtcctttcttcaccctttactgatttaatacagaataacaatcttattttctatttgataactatggtatcatattgggttactgtataaggaaaatggcaggggagttgtgggaagcttgtctttacaaaatataattgattaagatatgtcaagacctacattgtctaagcaccggcaaattaaaatgtcgagaatcacttcagtcaaaaacctttatattctgttcttaataatgtttgtgccaacctatatcccatgtaagggatctggggaggaggcatgtgtctacaaccatacctttttgcactatgggcactaaccaccctgaaacttcctgcggtagctccctcccttcagagttacatcattatcctgactctgtgtaggtaaatttccgtgaaatttttgtacaaaaaaaaggtaatgaaagaacgttgcaaagatcatctgcattataatgagttgatgctgttctcactcctctcttggaattgtgctggccccttagtctacaataaactgtgccaattaaaaacctaaggctaaaactgaaagccctttgatggggtcttaactcatatcagtcatttgggcttctctgatcctgaggctaagaaaggggaagagaccctcaggaggcagcttccactccagggctcttgatctctgctggattgggggtggccacctcagaaacttccaccctcatgactggaatggaagaggggaccgagagcctcacaatctcggagagggaggagaaattcttaaaaacagctgctctcctgcgcccagcttcacaggcagccctgcccctttctcctcaccagcatggtacctgcccttactgctagagcagctgcttgtagagggacattccctccttcccagttttaactggtggaccacagtggggggaaaaacattcaagcgatataaagacacttgggctctttgcagatgcctatacttccaacactaccatgtccacaaaccaccctgggggagggcccttccaaagggaggcttgctagtttcagcgtctagcagttgggtcctcacttttactccaattgtgaaaatagcccacgtaccctcgcagtgtccagtagggatcccagaggcacataaccaagaaaggattttgactttgtcacagtgactatttaaaataatctattcgaagtccaaaccaaacacaaagcctgtgatattttaggttattaaggtaactgctaatgaaggattttaaaaagtgtcctcaaaaaggacttagccccgggagttgtttataaaatttcccccacttgtatacagtgtgcactaaaagaaaatgtattttaatatctaatgcctgggctctgagcgtcatgcttcttggtggtaaacatgcagtcctgttcctaagtgactcagaggcatcagaatttctccacgttacccatctgcttggcactcggaactgagcgtgtgaaatccatagcgctgcccacaacctgttctcactgcttagctcccagctggattaaagacacctgctcaggcgggagagagagagagagagcgagcttttaccttggaaaaggtaaagatggaaatgtacaccaaaaaagacaatttttacatttaatggaacattctttttttttacaagtatatttttctactgatagtttcagaacactaatcttatattcactctaatcttaaacatgtttctttaaatatttataaggcagtttattacagaatattttcatgcaatcatgtgcacattattggtagcaaacatagtatatcctttagtactttagcatatttttgttaaaatacttttaatggtaagaaatgaacttgaggtcccaggaggttttgttgccttttcattgattagagacaataaatatcttgtaacttcctaaccagatctgagctgtgctcacaataataataatgaaatcagattctttgatgctggactcaggagggaaatcattagccaactgttgacttacttatagctagatgtctatgtgagaaagtataatatatatatatacacatatatatgacatgtaagagtcacttttatttatctgtctttgttcacttatgaagccggtaactgcagcagtatgttggtgatgtcatgatgcacagaagtcccatgtggagtgtttttcccacactgacaacttggcctcctttctgtgtgttcagtctgttgtctgaactaacactcccgcgagcactatactctttatactctgatccccctagttcatcttaaatttgtctgtggccctggcaagatagcgtacacaagattccatgactccagagcatcttgaagaaacatacatattttgaaagaggggaaatgtagcagatagttcacaagctgcgggttgtagctaaatattccatttctttgaaatcatgtttctaaattctttaccatcagaaagaaaaggagtgtcatacactttcaagggaaggcttggtctgcgttttctgtgtttggattatttttatactttgctgatctttaagctatccatgggggaaattttataccaacgagttaataattctcattcatcgtttacacaatgtaacatgtgtcatactggggccagcgagatggctcagtaggtaaaggtgcttgatgctaagcccggcagcctgtgtttcatctacaggatgcacaacataaaagaaaagatctgattcccacaggttctcttctgacctacacacacacacactaaaataacatttaaaaatatgtgccaaattatatttgttcgggtgccaccttccaccagcttaccactacggtagaactgtcaaattcatctccctgaatttgtcttaaaggggtgtccatgcacaggcccaagagtcacctccaatgaaataaatgtaatactgaagtatgccatgatgtttgttgttttctttcatcgtaagcctgtaagcaggaaaaatacgtcaaatcagatagaatagagcatttaccagtggtcgatggcctggtcagtcctgtgccgggtgacttaggaccaggcacgtcagctctctgagcctccccttcccttgttgtcacaagggaatagaagcagaagaagctgagagcctccctattcccagatgccctggtggaatgacctgcctctctgccgtttctgccaacgtgttggtgctataagctgcttcaaataccagtttgtctgtagtgtgtactcacctaatcacttgttatccagtgcctgttctaggtttatggacttaactatttctgtgatgtttcatttttagccatgttaactcctaacacatattctcttatgtctcagtaaagtttcatttgataagttgttgagattctgttatttgataatattcttcggctgtccatccagcatcttaatcactttaaaactgtgattgttatttgcaactctgttctttggaaagaataaaagcattttttttcacttgctaacatgctcacaaatgtgagagaagagtcattaaaagctttacttactgggtcagtgcgtcattgactcctttctgtgttttgcccaataaattaataaaagaccaaaaaaaaaaaaaaaaaaaaaaaaaaaaa

SEQ ID NO: 3 - DNA, 4175 bp, Mus musculus
  cDNA sequence of mouse G-gamma 12 protein subunit, variant 2
gcagcggcggcggcggcgacgacggcgaagagttcatatgggaaggactttggggtgagcatcttctctatttccagctggcttttctgattcaccccaccatttaaaacctggaggcactgggccacacaaagccttgctgattttcagaaagaagactcatcaaagatgtccagcaagacggcaagcaccaacagcatagcccaagccaggagaactgtgcagcagctgagattggaagcctccatcgaaagaataaaggtctcaaaagcatcagcagacctgatgtcatactgtgaggagcatgcccggagcgaccccctgctgatgggcataccgacctcagaaaacccgttcaaggataagaagacctgcatcatcttatagtggaccaggaagcgccccttgcctcttaacgcaaaccacagcagcaacctgaagggattccttcagcttacctggtaaccacagctagtaactaaaacacccttctctcggaataatagaccctgaagtctctctttttcaagttgtcctttcttcaccctttactgatttaatacagaataacaatcttattttctatttgataactatggtatcatattgggttactgtataaggaaaatggcaggggagttgtgggaagcttgtctttacaaaatataattgattaagatatgtcaagacctacattgtctaagcaccggcaaattaaaatgtcgagaatcacttcagtcaaaaacctttatattctgttcttaataatgtttgtgccaacctatatcccatgtaagggatctggggaggaggcatgtgtctacaaccatacctttttgcactatgggcactaaccaccctgaaacttcctgcggtagctccctcccttcagagttacatcattatcctgactctgtgtaggtaaatttccgtgaaatttttgtacaaaaaaaaggtaatgaaagaacgttgcaaagatcatctgcattataatgagttgatgctgttctcactcctctcttggaattgtgctggccccttagtctacaataaactgtgccaattaaaaacctaaggctaaaactgaaagccctttgatggggtcttaactcatatcagtcatttgggcttctctgatcctgaggctaagaaaggggaagagaccctcaggaggcagcttccactccagggctcttgatctctgctggattgggggtggccacctcagaaacttccaccctcatgactggaatggaagaggggaccgagagcctcacaatctcggagagggaggagaaattcttaaaaacagctgctctcctgcgcccagcttcacaggcagccctgcccctttctcctcaccagcatggtacctgcccttactgctagagcagctgcttgtagagggacattccctccttcccagttttaactggtggaccacagtggggggaaaaacattcaagcgatataaagacacttgggctctttgcagatgcctatacttccaacactaccatgtccacaaaccaccctgggggagggcccttccaaagggaggcttgctagtttcagcgtctagcagttgggtcctcacttttactccaattgtgaaaatagcccacgtaccctcgcagtgtccagtagggatcccagaggcacataaccaagaaaggattttgactttgtcacagtgactatttaaaataatctattcgaagtccaaaccaaacacaaagcctgtgatattttaggttattaaggtaactgctaatgaaggattttaaaaagtgtcctcaaaaaggacttagccccgggagttgtttataaaatttcccccacttgtatacagtgtgcactaaaagaaaatgtattttaatatctaatgcctgggctctgagcgtcatgcttcttggtggtaaacatgcagtcctgttcctaagtgactcagaggcatcagaatttctccacgttacccatctgcttggcactcggaactgagcgtgtgaaatccatagcgctgcccacaacctgttctcactgcttagctcccagctggattaaagacacctgctcaggcgggagagagagagagagagcgagcttttaccttggaaaaggtaaagatggaaatgtacaccaaaaaagacaatttttacatttaatggaacattctttttttttacaagtatatttttctactgatagtttcagaacactaatcttatattcactctaatcttaaacatgtttctttaaatatttataaggcagtttattacagaatattttcatgcaatcatgtgcacattattggtagcaaacatagtatatcctttagtactttagcatatttttgttaaaatacttttaatggtaagaaatgaacttgaggtcccaggaggttttgttgccttttcattgattagagacaataaatatcttgtaacttcctaaccagatctgagctgtgctcacaataataataatgaaatcagattctttgatgctggactcaggagggaaatcattagccaactgttgacttacttatagctagatgtctatgtgagaaagtataatatatatatatacacatatatatgacatgtaagagtcacttttatttatctgtctttgttcacttatgaagccggtaactgcagcagtatgttggtgatgtcatgatgcacagaagtcccatgtggagtgtttttcccacactgacaacttggcctcctttctgtgtgttcagtctgttgtctgaactaacactcccgcgagcactatactctttatactctgatccccctagttcatcttaaatttgtctgtggccctggcaagatagcgtacacaagattccatgactccagagcatcttgaagaaacatacatattttgaaagaggggaaatgtagcagatagttcacaagctgcgggttgtagctaaatattccatttctttgaaatcatgtttctaaattctttaccatcagaaagaaaaggagtgtcatacactttcaagggaaggcttggtctgcgttttctgtgtttggattatttttatactttgctgatctttaagctatccatgggggaaattttataccaacgagttaataattctcattcatcgtttacacaatgtaacatgtgtcatactggggccagcgagatggctcagtaggtaaaggtgcttgatgctaagcccggcagcctgtgtttcatctacaggatgcacaacataaaagaaaagatctgattcccacaggttctcttctgacctacacacacacacactaaaataacatttaaaaatatgtgccaaattatatttgttcgggtgccaccttccaccagcttaccactacggtagaactgtcaaattcatctccctgaatttgtcttaaaggggtgtccatgcacaggcccaagagtcacctccaatgaaataaatgtaatactgaagtatgccatgatgtttgttgttttctttcatcgtaagcctgtaagcaggaaaaatacgtcaaatcagatagaatagagcatttaccagtggtcgatggcctggtcagtcctgtgccgggtgacttaggaccaggcacgtcagctctctgagcctccccttcccttgttgtcacaagggaatagaagcagaagaagctgagagcctccctattcccagatgccctggtggaatgacctgcctctctgccgtttctgccaacgtgttggtgctataagctgcttcaaataccagtttgtctgtagtgtgtactcacctaatcacttgttatccagtgcctgttctaggtttatggacttaactatttctgtgatgtttcatttttagccatgttaactcctaacacatattctcttatgtctcagtaaagtttcatttgataagttgttgagattctgttatttgataatattcttcggctgtccatccagcatcttaatcactttaaaactgtgattgttatttgcaactctgttctttggaaagaataaaagcattttttttcacttgctaacatgctcacaaatgtgagagaagagtcattaaaagctttacttactgggtcagtgcgtcattgactcctttctgtgttttgcccaataaattaataaaagaccaaaaaaaaaaaaaaaaaaaaaaaaaaaaa

SEQ ID NO: 4 - protein, 72 aa, Homo sapiens
  Amino acid sequence of human G-gamma 12 protein subunit
MSSKTASTNNIAQARRTVQQLRLEASIERIKVSKASADLMSYCEEHARSDPLLIGIPTSENPFKDKKTCIIL

SEQ ID NO: 5 - protein, 8 aa, Artificial Sequence
  Description of Artificial Sequence General epitope tag
DYKDDDDK

(19) United States
(12) Patent Application Publication
(10) Pub. No.: US 2003/0118990 A1
(43) Pub. Date: Jun. 26, 2003

(54) Novel estrogen-regulated G protein gamma subunit: compositions and methods of use

(75) Inventors: Alan R. Brooks, Clayton, CA (US); Gary G. Deng, Benicia, CA (US); Gabor M. Rubanyi, Alamo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Schering Aktiengesellschaft

(21) Appl. No.: 09/803,472

(22) Filed: Mar. 9, 2001

Related U.S. Application Data
(60) Provisional application No. 60/188,460, filed on Mar. 10, 2000.

(51) Int. Cl. C12Q 1/68; G01N 33/53; G01N 33/567; C12P 21/02; C12N 5/06; C07K 14/72

(52) U.S. Cl. 435/6; 435/7.2; 435/69.1; 435/320.1; 435/325; 530/350; 514/12; 536/23.5

(57) ABSTRACT

The present invention provides novel nucleic acid and protein sequences for an estrogen-regulated GTP-binding protein gamma-12 subunit protein, as well as methods for using the sequences for modulating the effects of estrogen in mammalian cells, methods of detecting estrogen signaling in cells, and methods of identifying compounds capable of acting as an estrogen receptor agonist or antagonist.

### CROSS REFERENCE TO RELATED APPLICATION

This application claims benefit of U.S. Provisional Application No. 60/188,460, filed Mar. 10, 2000, which application is incorporated herein by reference for all purposes.

### STATEMENT AS TO RIGHTS TO INVENTIONS MADE UNDER FEDERALLY SPONSORED RESEARCH AND DEVELOPMENT

Not Applicable.

### BACKGROUND OF THE INVENTION

The ovarian sex steroid hormone estrogen has a broad spectrum of biological functions including inhibiting the development of atherosclerosis, osteoporosis, and neurological disorders including Alzheimer's Disease and Parkinson's Disease. Estrogen has also been associated with breast and other cancers. Despite the importance of estrogen and estrogen signaling in these and other diseases, the molecular mechanisms of estrogen action are largely unknown, and the ability to modulate estrogen signaling remains poorly developed.

Estrogen regulates the expression of many genes by binding to its two known nuclear receptors, ERα and ERβ. These two receptor types are encoded by distinct genes, and have distinct ligand affinity and tissue distribution (see, e.g., Kuiper et al., (1997) Endocrinology 138:863-870). The importance of estrogen receptor α has been demonstrated by the generation of a knockout mouse, called ERKO. Both male and female ERKO mice are sterile and display a variety of phenotypic effects including decreased bone density, defects in their reproductive tissues, and decreased likelihood of oncogene-induced cancer (see, e.g., Couse et al., (1999) Endocr. Rev. 20:358-417; Korach (1994) Science 266:1524-1527; Bocchinfuso et al., (1999) Cancer Res. 59:1869-1876). Mice lacking estrogen receptor β are fertile, but females display decreased ovarian activity, leading to decreased litter sizes (Krege, et al. (1998) Proc. Natl. Acad. Sci. USA 95:15677-15682). Mice lacking both α and β receptors are infertile and display an ovarian phenotype that is distinct from that of either receptor knockout alone (Couse et al., (1999) Science 286:2328-2331).

GTP-binding proteins (G-proteins) are a family of proteins that transduce signals from numerous cell surface receptors to downstream intracellular effector molecules. G-proteins are heterotrimeric proteins consisting of the α, β and γ subunits, the latter two being tightly associated under physiological conditions. Each subunit is encoded by a separate gene. Upon binding of a ligand to a receptor, the GDP molecule bound to the alpha subunit is exchanged for a GTP molecule resulting in the dissociation of the α subunit from the β and γ subunits. Many studies have shown that receptor stimulation promotes the dissociation of the G protein into a GTP-bound α subunit and a βγ dimer, both of which independently regulate intracellular effectors. Many studies have shown that the βγ dimer regulates adenylyl cyclase, phospholipase C, phosphatidylinositol 3-kinase, receptor kinase, K⁺ channels, and Ca²⁺ channels. The βγ complex was also found to be involved in the stimulation of mitogen-activated protein kinase.

Thus, there is a need in the art for new approaches to understanding and modulating estrogen signaling in animals. The present invention addresses this and other needs.

### SUMMARY OF THE INVENTION

The present invention provides novel nucleic acid and protein sequences for an estrogen-regulated GTP-binding protein gamma-12 protein (mGγ12), as well as methods for using the sequences for modulating the effects of estrogen in mammalian cells, methods of detecting estrogen signaling in cells, and methods of identifying compounds capable of acting as an estrogen receptor agonist or antagonist.

In one aspect, the present invention provides an isolated nucleic acid encoding an estrogen-regulated GTP-binding protein gamma-12 protein, the protein comprising the amino acid sequence of SEQ ID NO: 1. In one embodiment, the nucleic acid comprises a nucleotide sequence that is at least about 70% identical to SEQ ID NO: 2 or 3. In another embodiment, the nucleic acid comprises the nucleotide sequence of SEQ ID NO: 2 or 3. In yet another embodiment, the nucleic acid hybridizes under moderately stringent wash conditions to a nucleic acid comprising the nucleotide sequence of SEQ ID NO: 2 or 3. In another embodiment, the nucleic acid hybridizes under stringent wash conditions to a nucleic acid comprising the nucleotide sequence of SEQ ID NO: 2 or 3. In some embodiments, the nucleic acid is from a mouse.

In another aspect, the present invention provides an expression cassette comprising the nucleic acid. The present invention further provides an isolated eukaryotic cell comprising the expression cassette.

In yet another aspect, the present invention provides an isolated estrogen-regulated GTP-binding protein gamma-12 protein, the protein comprising the amino acid sequence of SEQ ID NO: 1. In one embodiment, the protein is from a mouse.

The present invention also provides antibodies that selectively bind to an estrogen-regulated GTP-binding protein gamma-12 protein comprising the amino acid sequence of SEQ ID NO: 1 and that do not bind to the estrogen-regulated GTP-binding protein gamma-12 subunit protein having the amino acid sequence of SEQ ID NO: 4.

In another aspect, the present invention provides a method of modulating the effects of estrogen in a mammalian cell, the method comprising modulating the level of expression or activity of an estrogen-regulated GTP-binding protein gamma-12 protein having the amino acid sequence of SEQ ID NO: 1.

In one embodiment, the level of expression is modulated by introducing a polynucleotide into the cell, whereby the presence or expression of the polynucleotide modulates the level of expression of the estrogen-regulated GTP-binding protein gamma-12 protein in the cell. In another embodiment, the polynucleotide encodes a full length estrogen-regulated GTP-binding protein gamma-12 protein, wherein expression of the polynucleotide increases the level of expression of the estrogen-regulated GTP-binding protein gamma-12 protein in the cell. In another embodiment, the polynucleotide is an antisense sequence, wherein the presence or expression of the polynucleotide decreases the level of expression of estrogen-regulated GTP-binding protein gamma-12 protein in the cell. In yet another embodiment, a compound is administered to the cell, whereby the level of the expression or activity of the estrogen-regulated GTP-binding protein gamma-12 protein is modulated. In another embodiment, the effects of estrogen are mediated by an estrogen receptor α.

In another embodiment, the cell is present in a mammal. In another embodiment, the level of expression or activity of the estrogen-regulated GTP-binding protein gamma-12 protein is increased, whereby the development of atherosclerosis or osteoporosis in the mammal is inhibited.

In another aspect, the present invention provides a method of detecting the presence of estrogen signaling in a mammalian cell, the method comprising detecting the expression of an estrogen-regulated GTP-binding protein gamma-12 protein encoding nucleic acid in the cell, the protein comprising the amino acid sequence of SEQ ID NO: 1.

In one embodiment, the presence of estrogen signaling is used in order to determine the tissue-specific distribution of estrogen signaling in a mammal. In another embodiment, the estrogen signaling is mediated by an estrogen receptor α. In another embodiment, the expression of the nucleic acid in the cell is detected by detecting the expression or activity of the encoded estrogen-regulated GTP-binding protein gamma-12 protein in the cell. In yet another embodiment, the expression of the nucleic acid in the cell is detected by detecting the level of estrogen-regulated GTP-binding protein gamma-12 protein mRNA in the cell.

Finally, the present invention also provides a method of identifying a compound capable of acting as an estrogen-receptor agonist or antagonist, the method comprising: (1) contacting a cell comprising an estrogen receptor with the compound; and (2) detecting the functional effect of the compound on the cell, wherein an increase in the level of estrogen regulated GTP-binding protein gamma-12 mRNA, protein, or protein activity in the cell indicates that the compound is capable of acting as an estrogen receptor agonist, and a decrease in the level of estrogen regulated GTP-binding protein gamma-12 mRNA, protein, or protein activity in the cell indicates that the compound is capable of acting as an estrogen receptor antagonist.

In one embodiment, the estrogen receptor is an estrogen receptor α. In another embodiment, the mRNA is at least about 70% identical to the sequence shown as SEQ ID NO: 2 or 3, and the protein comprises the sequence of SEQ ID NO: 1.

### BRIEF DESCRIPTION OF THE DRAWINGS

FIG. 1 provides the amino acid sequence for the mouse Gγ12 protein (mGγ12) (SEQ ID NO: 1).

FIG. 2 provides the nucleotide sequence for a cDNA encoding the mouse Gγ12 variant 1 (SEQ ID NO: 2).

FIG. 3 provides the nucleotide sequence for a cDNA encoding the mouse Gγ12 variant 2 (SEQ ID NO: 3).

FIG. 4 provides an amino acid sequence for human Gγ12 (SEQ ID NO: 4).

FIG. 5 illustrates the domain structure for Gγ12. The domain structure was analyzed with HMM using the translated amino acid sequences.

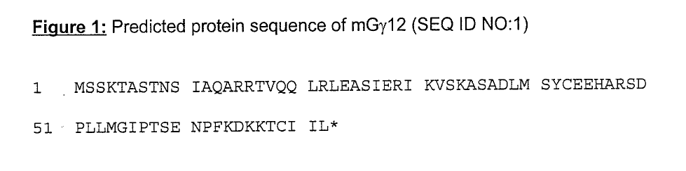

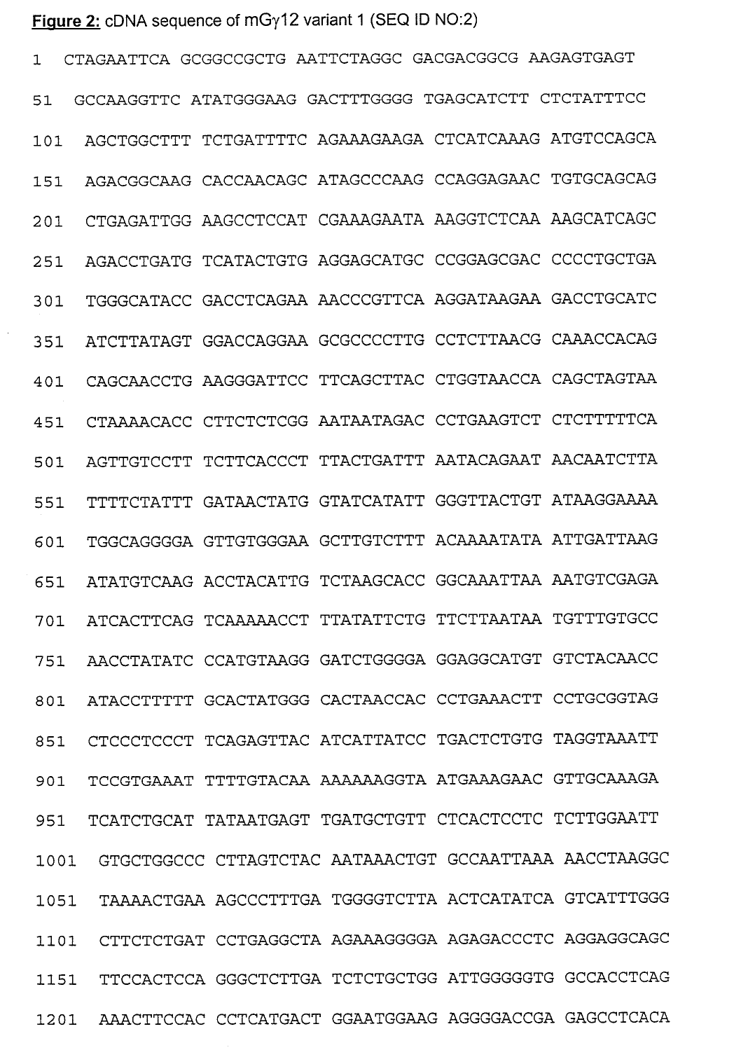

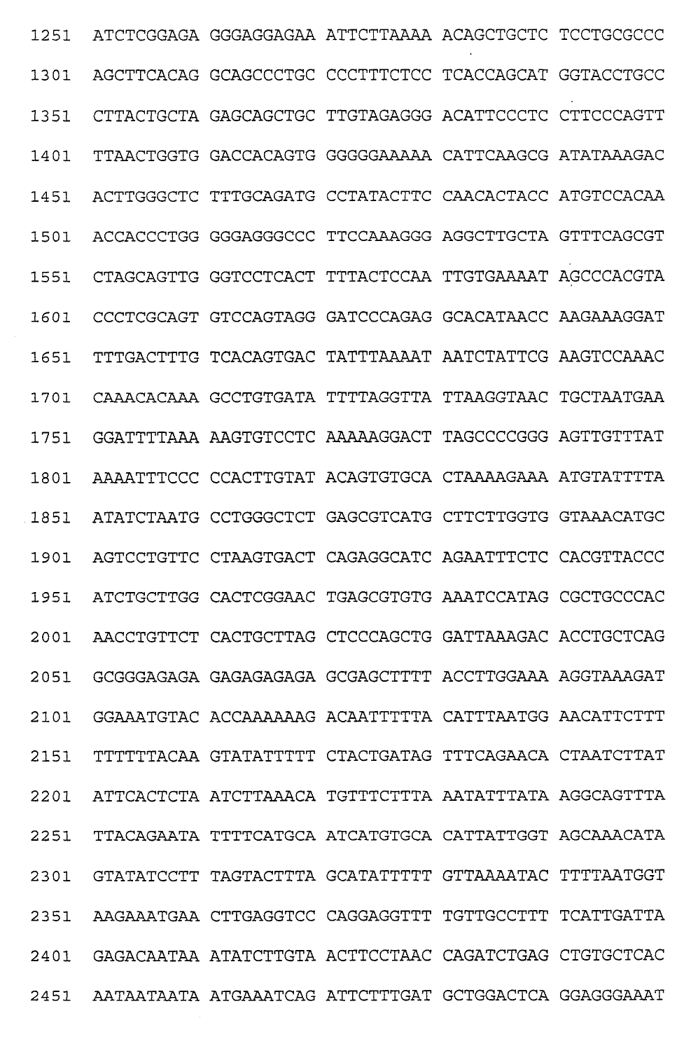

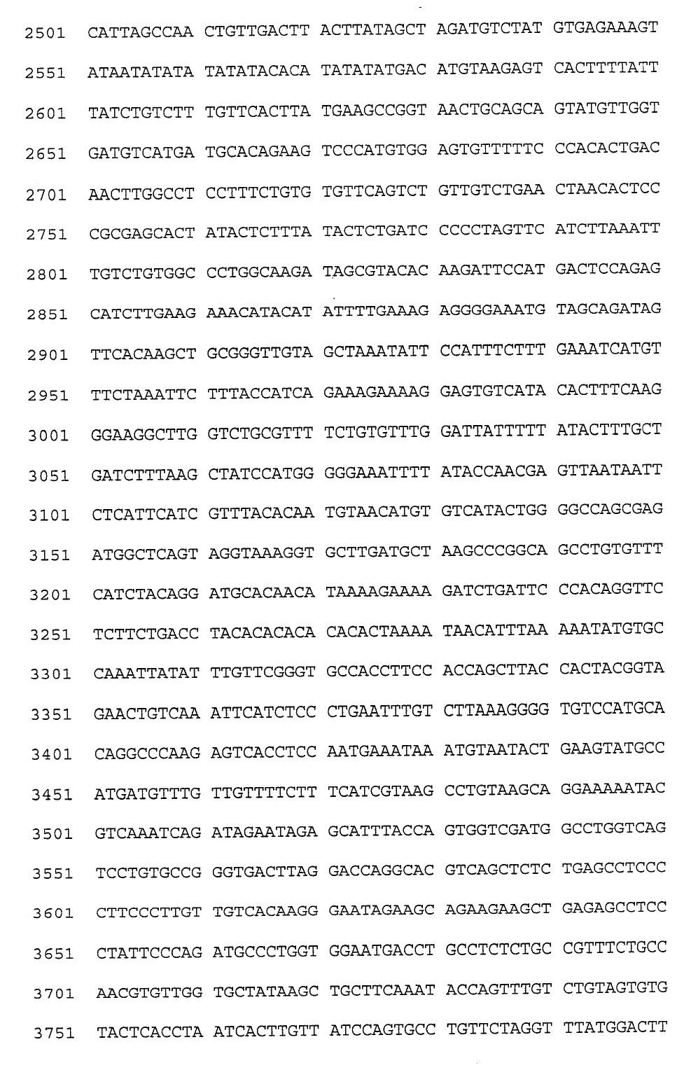

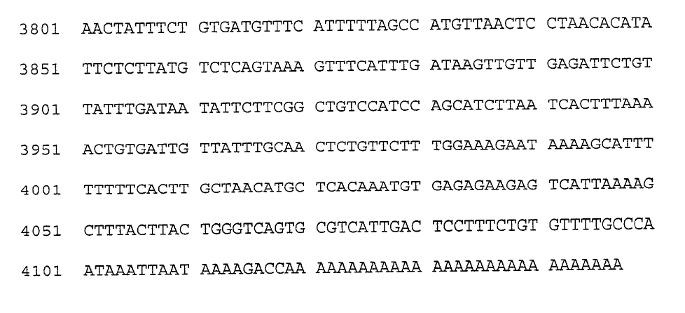

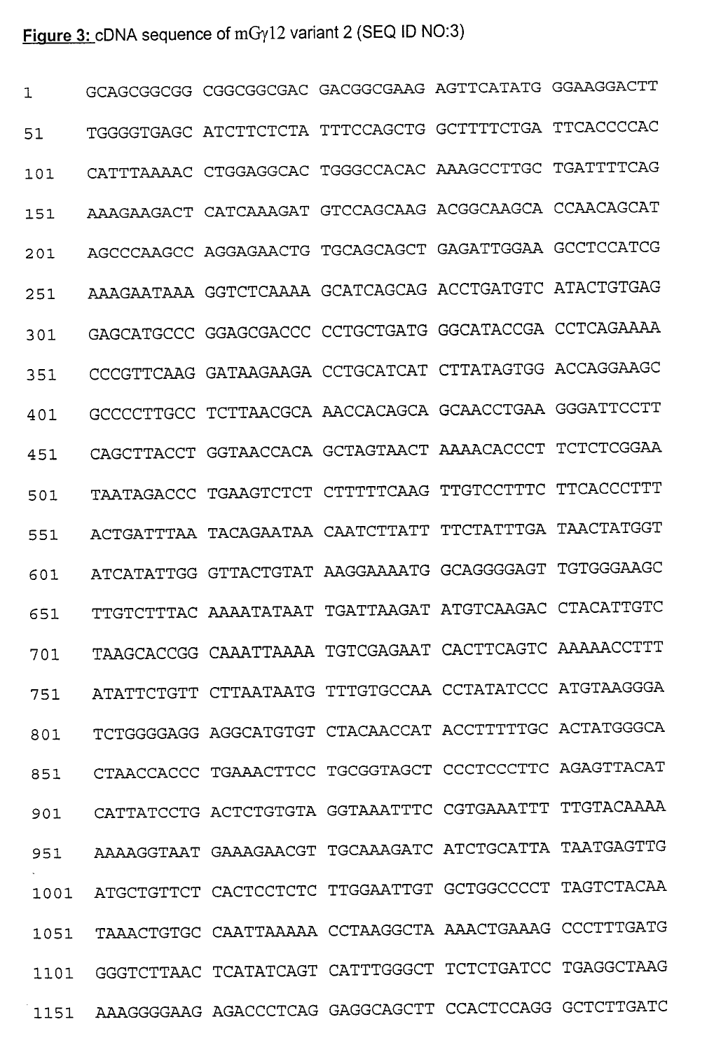

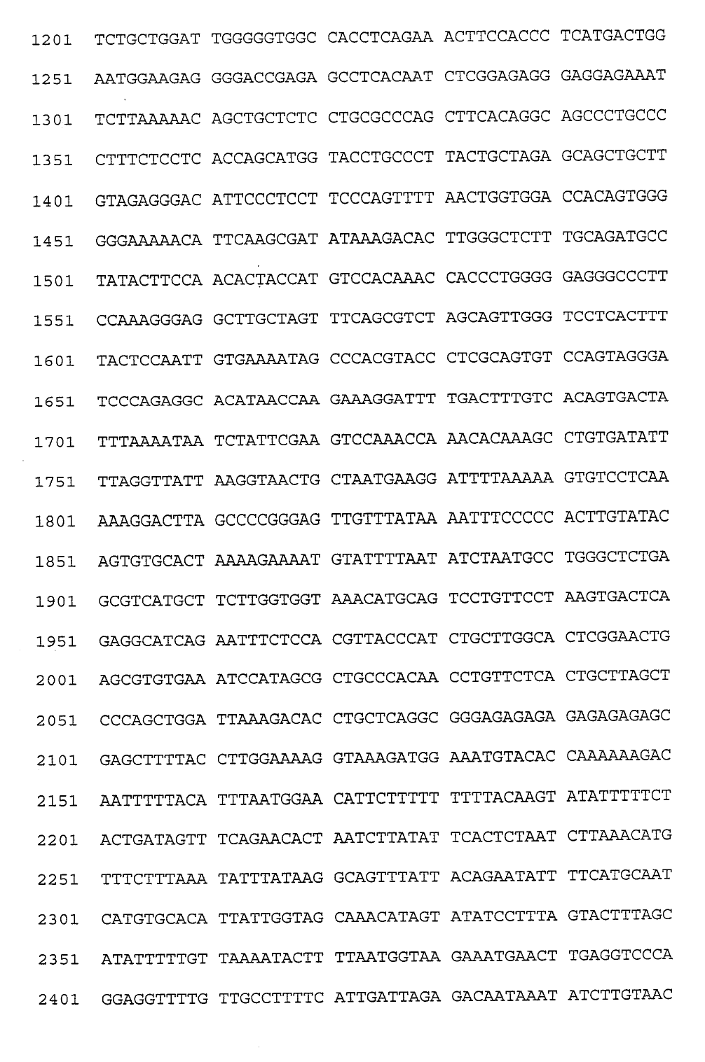

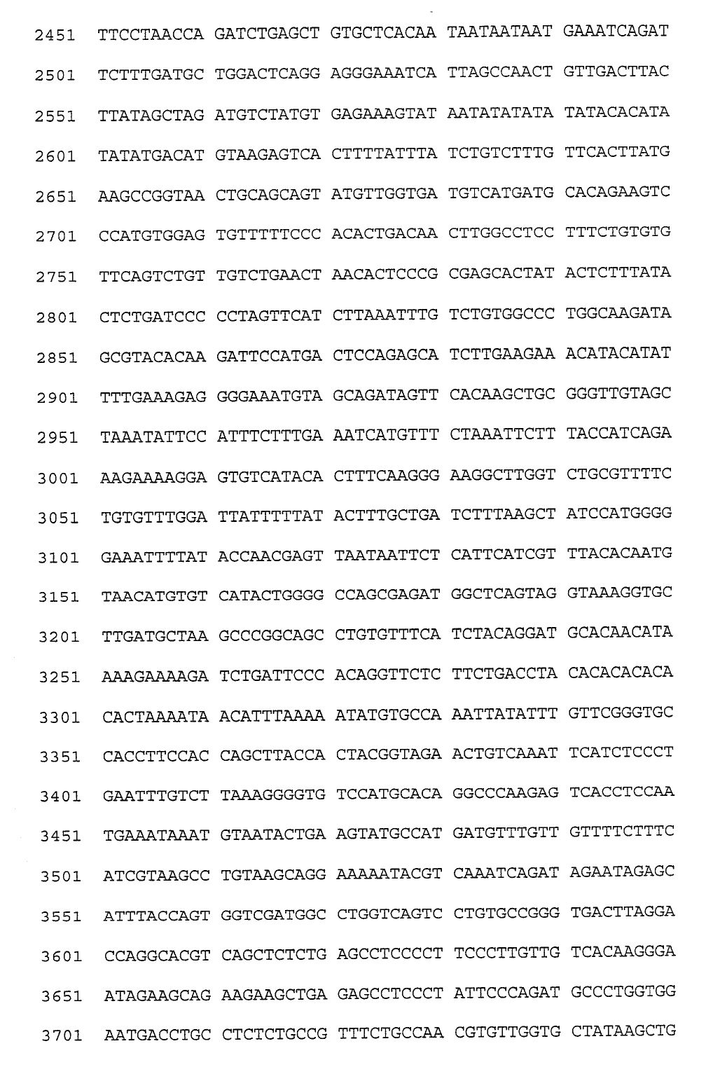

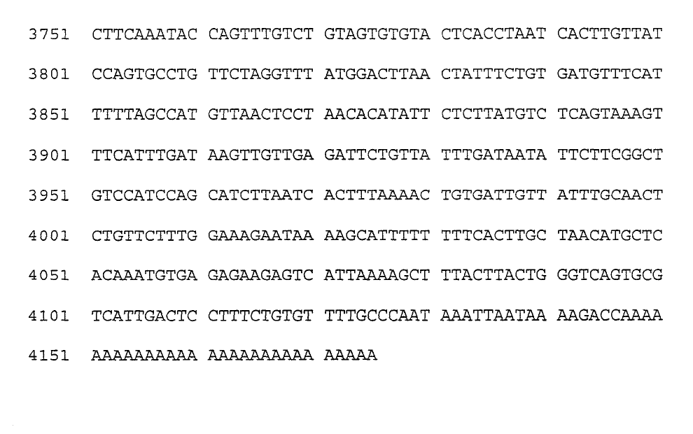

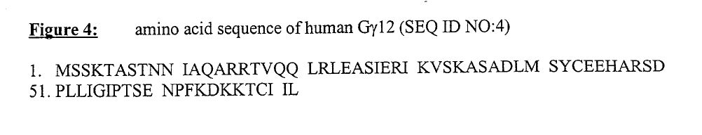

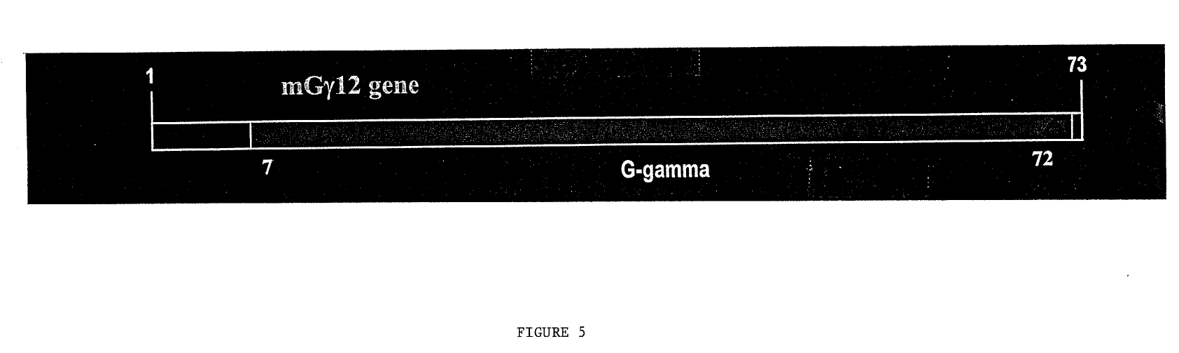

### DETAILED DESCRIPTION OF THE INVENTION AND PREFERRED EMBODIMENTS

### I. Introduction

The present invention provides nucleic acids and polypeptides for estrogen-regulated GTP-binding protein gamma 12 subunit protein (Gγ12), and in particular for a novel mouse estrogen-regulated GTP-binding protein gamma 12 subunit protein (mGγ12). mGγ12 is dramatically upregulated by estrogen, indicating that mGγ12, and Gγ12 proteins in general, are molecular mediators of the in vivo function of estrogen. Accordingly, the effects of estrogen can be modulated in cells by modulating the expression or activity of Gγ12, and, in addition, the presence, level, or tissue distribution of estrogen signaling can be detected by detecting Gγ12 expression or activity. Gγ12 can also be used as a marker gene for the investigation of tissue-specific and estrogen receptor-specific ligands, such as estrogen receptor agonists and antagonists.

Gγ12 sequences are provided. For example, two variants of a mouse Gγ12 (mGγ12) cDNA, shown as SEQ ID NOs :2 and 3, are provided. These cDNAs encode a distinct protein, shown as SEQ ID NO: 1. In addition, the amino acid sequence of a human Gγ12 protein is also provided in SEQ ID NO: 4.

Modulators, recombinant forms, derivatives, variants, or fragments of the herein-described Gγ12s can be used to enhance or inhibit estrogen signaling in cells, and can therefore be useful for the treatment of a wide variety of estrogen-related diseases. For example, estrogen signaling can be enhanced to treat diseases including, but not limited to, osteoporosis, cardiovascular diseases, Alzheimer's Disease, or Parkinson's Disease. Alternatively, estrogen signaling can be inhibited to treat, e.g., breast or other cancers.

In numerous embodiments, the present invention provides methods of screening for modulators, e.g., activators, inhibitors, stimulators, enhancers, etc., of Gγ12 nucleic acids and proteins. Such modulators can affect Gγ12 activity in any of a number of ways, e.g., by modulating Gγ12 transcription, translation, phosphorylation, RNA or protein stability, by altering the binding of Gγ12 to heterologous proteins or other molecules, or by affecting Gγ12 protein activity. In preferred embodiments, modulators that increase or decrease Gγ12 activity or levels are used to treat any of the above-recited estrogen-related diseases or conditions.

In one embodiment, compounds are screened, e.g., using high throughput screening (HTS), to identify those compounds that can bind to and/or modulate the activity of an isolated Gγ12 polypeptide or fragment thereof. In another embodiment, Gγ12 proteins are recombinantly expressed in cells, and potential modulators of Gγ12 are assayed by detecting the presence or activity of Gγ12 in the cells.

In numerous embodiments, a Gγ12 polynucleotide or polypeptide is introduced into a cell, in vivo or ex vivo, and the Gγ12 activity in the cell is thereby modulated. For example, a polynucleotide encoding a full length Gγ12 polypeptide is introduced into a population of cells, thereby increasing the level or activity of Gγ12 in the cells. Alternatively, an antisense-, ribozyme-, or dominant-negative-encoding polynucleotide can be introduced into a population of cells, thereby inhibiting the Gγ12 levels and/or activity in the cells.

The present invention also provides methods for detecting Gγ12 nucleic acid and protein expression, allowing investigation into estrogen-mediated signaling and transcription, e.g., through an estrogen receptor α, and allowing the specific identification, in vitro or in vivo, of estrogen responsive cells. Gγ12 polypeptides can also be used to generate monoclonal and polyclonal antibodies useful for identifying estrogen responsive cells, particularly cells responsive to estrogen through an estrogen receptor α. Gγ12 expression can be identified using techniques such as reverse transcription and amplification of mRNA, isolation of total RNA or poly A⁺ RNA, northern blotting, dot blotting, in situ hybridization, RNase protection, S1 digestion, probing DNA microchip arrays, western blots, and the like.

Functionally, Gγ12 nucleic acids encode members of the GTP-binding protein family that are strongly upregulated by estrogen signaling. Related Gγ12 genes from different species share at least about 80% nucleotide sequence similarity or identity over a region of at least about 50 nucleotides in length, optionally 65 or more nucleotides in length, to SEQ ID NO: 2 or 3, or encode polypeptides sharing at least about 80% amino acid sequence similarity or identity over an amino acid region at least about 25 amino acids in length, optionally 50 to 70 amino acids in length, to SEQ ID NO: 1. Preferably, the Gγ12 polypeptide comprises from about 65-75 amino acids. More preferably, the Gγ12 polypeptide comprises about 72 amino acids.

The present invention also provides polymorphic variants of the mGγ12 protein depicted in SEQ ID NO: 1.

Specific regions of the Gγ12 nucleotide and amino acid sequences may be used to identify polymorphic variants, interspecies homologs, and alleles of Gγ12 genes. This identification can be made in vitro, e.g., under stringent hybridization conditions, or by PCR and sequencing, or by using the sequence information in a computer system for comparison with other nucleotide sequences. Typically, identification of polymorphic variants and alleles of a Gγ12 protein is made by comparing an amino acid sequence of about 25 amino acids or more, e.g., 50-70 amino acids. Amino acid identity of approximately at least 80% or above, optionally 85%-95% or above, typically demonstrates that a protein is a polymorphic variant, interspecies homolog, or allele of a given Gγ12 protein. Sequence comparison can be performed using any of the sequence comparison algorithms discussed below. Antibodies that bind specifically to mGγ12 polypeptides or a conserved region thereof can also be used to identify alleles, interspecies homologs, and polymorphic variants.

Polymorphic variants, interspecies homologs, and alleles of the herein-described Gγ12s are confirmed by examining, e.g., expression or activity of the putative homolog in response to estrogen. Typically, an mGγ12 polypeptide having an amino acid sequence of SEQ ID NO: 1 is used as a positive control in comparison to the putative Gγ12 protein to demonstrate the identification of a polymorphic variant or allele of the mGγ12 gene or protein.

Nucleotide and amino acid sequence information for the present mGγ12 sequences may also be used to construct models of Gγ12 polypeptides in a computer system. These models are subsequently used to identify compounds that can activate or inhibit Gγ12 proteins. Such compounds can be used to investigate the role of Gγ12 genes in estrogen mediated signaling, and to treat any of a number of estrogen related diseases or conditions.

The present invention also provides assays, preferably high throughput assays, to identify compounds or other molecules that interact with and/or modulate a Gγ12.

The present invention also provides methods to treat diseases or conditions associated with estrogen signaling. For example, Gγ12 activity and/or expression can be altered in cells of a patient to treat or prevent diseases and conditions including, but not limited to, atherosclerosis, osteoporosis, Alzheimer's Disease, Parkinson's Disease, and breast cancer.

Transgenic animals and cells lacking one or more Gγ12 alleles, or containing altered forms of a Gγ12 are also provided, as are kits for using the herein-disclosed polynucleotides and polypeptides and for practicing the herein-disclosed methods.

### II. Definitions

As used herein, the following terms have the meanings ascribed to them below unless specified otherwise.

As used herein, “Gγ12” refers to a GTP-binding protein gamma 12 subunit that has at least 80% sequence identity to the amino acid sequence of SEQ ID NO: 1, or any derivative, homolog, or fragment thereof, or to any nucleic acid encoding such a protein, derivative, homolog, or fragment thereof. “mGγ12” refers to a mouse GTP-binding protein gamma 12 subunit as shown in SEQ ID NO: 1, or any derivative, homolog, or fragment thereof, or to any nucleic acid encoding such a protein, derivative, homolog, or fragment thereof. Gγ12 proteins or derivatives can be expressed in any cell type, including any eukaryotic or prokaryotic cell, or synthesized in vitro. Typically, Gγ12 nucleic acids encode active GTP-binding protein gamma-12 subunits that bind to GTP-binding protein beta and/or alpha subunits. It will be recognized that derivatives, homologs, and fragments of Gγ12 can readily be used in the present invention. Such Gγ12 variants can comprise any one or more fragments of, e.g., the polypeptide shown as SEQ ID NO: 1, or multiple copies of any one or more fragments, or any number of fragments in novel combinations with each other or with other proteins or protein fragments.

The term “mGγ12” also refers to polymorphic variants, alleles, mutants, and interspecies homologs that: (1) have about 60% amino acid sequence identity, optionally about 75, 80, 85, 90, or 95% amino acid sequence identity to SEQ ID NO: 1 over a window of about 25 amino acids, optionally 50-100 amino acids; (2) specifically bind to antibodies raised against an immunogen comprising an amino acid sequence of SEQ ID NO: 1, and conservatively modified variants thereof; or (3) specifically hybridize (with a size of at least about 100, optionally at least about 500-1000 nucleotides) under stringent hybridization conditions to a sequence of SEQ ID NO: 2 or 3, and conservatively modified variants thereof.

“Biological sample,” as used herein, refers to a sample of biological tissue or fluid that contains one or more Gγ12 nucleic acids encoding one or more Gγ12 proteins. Such samples include, but are not limited to, tissue isolated from mammals, in particular, e.g., liver, thymus, spleen, kidney, placenta, lung, kidney, pancreas, prostate, testis, ovary, small intestine, colon, etc. Biological samples may also include sections of tissues such as frozen sections taken for histological purposes. A biological sample is typically obtained from eukaryotic organisms, such as insects, protozoa, birds, fish, reptiles, and preferably a mammal such as, e.g., a mouse, a rat, a cow, a dog, a guinea pig, or a rabbit, and can be from a primate such as, e.g., a chimpanzee or a human.

By “determining the functional effect” is meant assaying for a compound that modulates, e.g., increases or decreases, a parameter that is indirectly or directly under the influence of a Gγ12 polypeptide, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, changes in gene expression of Gγ12 or of any marker genes indicative of Gγ12 activity, and the like.

“Inhibitors,” “activators,” and “modulators” of Gγ12 genes or proteins are used to refer to inhibitory, activating, or modulating molecules identified using in vitro and/or in vivo assays for Gγ12 . Inhibitors are compounds that, e.g., bind to Gγ12 proteins, partially or totally block Gγ12 activity, downregulate Gγ12 expression or stability, or prevent Gγ12 binding to heterologous molecules, e.g., GTP-binding protein alpha and beta subunits. Activators are compounds that, e.g., bind to Gγ12 , stimulate Gγ12 activity, increase Gγ12 expression or stability, or facilitate Gγ12 binding to membranes or to any other protein or factor. Modulators may include genetically modified versions of Gγ12 proteins, e.g., dominant negative or activated forms of Gγ12. Such assays for inhibitors and activators are described below and include, e.g., expressing Gyγ12 proteins in cells, applying putative modulator compounds, and then determining the functional effects on Gγ12 activity. Samples or assays comprising Gγ12 polypeptides that are treated with a potential activator, inhibitor, or modulator are compared to control samples without the inhibitor, activator, or modulator to examine the effect of the candidate compound. Control samples (untreated with the compound) are assigned a relative Gγ12 activity value of 100%. Inhibition of a Gγ12 polypeptide is achieved when the activity value relative to the control is about 80%, optionally 50% or 25-0%. Activation of a Gγ12 polypeptide is achieved when the activity value relative to the control is 110%, optionally 150%, optionally 200-500%, or 1000-3000% higher.

The terms “isolated,” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated Gγ12 nucleic acid is separated from open reading frames that flank the Gγ12 gene and encode proteins other than Gγ12. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, or chemical means. For example, useful labels include ³²p, fluorescent dyes, electron-dense reagents, enzymes (e.g., as commonly used in an ELISA), biotin, digoxigenin, or haptens and proteins which can be made detectable, e.g., by incorporating a radiolabel into the peptide, or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 amino acids or nucleotides in length, or more preferably over a region that is 75-100 amino acids or nucleotides in length.

The term “similarity,” or percent “similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Such sequences are then said to be “substantially similar.” Optionally, this similarity exists over a region that is at least about 50 amino acids in length, or more preferably over a region that is at least about 75-100 amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc. Acids Res. 12:387-395).

Another example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1 989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C. Such washes can be performed for 5, 15, 30, 60, 120, or more minutes.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see, Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al. (1990) Nature 348:552-554).

For preparation of monoclonal or polyclonal antibodies, any technique known in the art can be used (see, e.g., Kohler and Milstein (1975) Nature 256:495-497; Kozbor et al. (1983) Immunology Today 4: 72; Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al. (1990) Nature 348:552-554; Marks et al. (1992) Biotechnology 10:779-783).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-Gγ12” antibody is an antibody or antibody fragment that specifically binds a polypeptide encoded by a Gγ12 gene, cDNA, or a subsequence thereof. An “anti-mGγ12” antibody is an antibody or antibody fragment that specifically binds a polypeptide encoded by an Gγ12 gene, cDNA, or a subsequence thereof and that does not bind a polypeptide encoded by a Gγ12 gene from a different species, such as, e.g., a human Gγ12.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a Gγ12 polypeptide from specific species such as rat, mouse, or human can be selected to obtain only those polyclonal antibodies that are specifically immunoreactive with the Gγ12 protein of interest (e.g., mGγ12) and not with other proteins, (e.g., with a human Gγ12) except for polymorphic variants and alleles of the Gγ12 protein of interest. This selection may be achieved by subtracting out antibodies that cross-react with Gγ12 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind” to a protein, as defined above.

By “host cell” is meant a cell that contains an expression vector and supports the replication or expression of the expression vector. Host cells may be prokaryotic cells such as, e.g., E coli, or eukaryotic cells such as, e.g., yeast, insect, amphibian, or mammalian cells such as CHO, HeLa and the like, e.g., cultured cells, explants, and cells in vivo.

### III. Manipulation and Detection off Gγ12 Nucleic Acids

In numerous embodiments of the present invention, nucleic acids encoding a Gγ12 polypeptide, including a full-length Gγ12 protein, or any derivative, variant, homolog, or fragment thereof, will be used. Such nucleic acids are useful for any of a number of applications, including for the production of Gγ12 protein, for diagnostic assays, for therapeutic applications, for Gγ12 specific probes, for assays for Gγ12 binding and/or modulating compounds, to identify and/or isolate Gγ12 homologs from other species, and other applications. In the different embodiments of the invention, the nucleic acids encoding the Gγ12 polypeptide can be from any mammal, including, in particular, e.g., a human, a mouse, etc.

A. General Recombinant DNA Methods

Numerous applications of the present invention involve the cloning, synthesis, maintenance, mutagenesis, and other manipulations of nucleic acid sequences that can be performed using routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage and Caruthers (1981) Tetrahedron Letts. 22:1859-1862, using an automated synthesizer, as described in Van Devanter et al (1984) Nucleic Acids Res. 12:6159-6168. Purification of oligonucleotides is by either native acrylamide gel electrophoresis or by anion-exchange HPLC as described in Pearson and Reanier (1983) J. Chrom. 255:137-149.

The sequence of the cloned genes and synthetic oligonucleotides can be verified after cloning using, e.g., the chain termination method for sequencing double-stranded templates of Wallace et al. (1981) Gene 16:21-26.

B. Isolating and Detecting Gγ12 Nucleotide Sequences

In numerous embodiments of the present invention, Gγ12 nucleic acids will be isolated and cloned using recombinant methods. Such embodiments are used, e.g., to isolate Gy 12 polynucleotides for protein expression or during the generation of variants, derivatives, expression cassettes, or other sequences derived from Gγ12, to monitor Gγ12 gene expression, for the isolation or detection of Gγ12 sequences in different species, for diagnostic purposes in a patient, e.g., to detect mutations in Gγ12. In one embodiment, the nucleic acids of the invention are from any mammal, including, in particular, e.g., a human, a mouse, etc.

Often, the nucleic acid sequences encoding Gγ12 proteins and related nucleic acid sequence homologs are cloned from cDNA and genomic DNA libraries by hybridization with probes, or isolated using amplification techniques with oligonucleotide primers. For example, Gγ12 sequences are typically isolated from mammalian nucleic acid (genomic or cDNA) libraries by hybridizing with a nucleic acid probe the sequence of which can be derived from specific Gγ12 genes, e.g., from SEQ ID NO: 2 or 3, or amplified using specific primers that can be designed from specific Gγ12 sequences, e.g., the sequences described in SEQ ID NO: 2 or 3, using standard techniques well-known to those of skill in the art. A suitable biological material from which RNA and cDNA for Gγ12 can be isolated includes a large variety of tissues such as, e.g., liver, thymus, spleen, kidney, placenta, lung, kidney, pancreas, prostate, testis, ovary, small intestine, colon, etc.

Amplification techniques using primers can also be used to amplify and isolate Gγ12 sequences from DNA or RNA (see, e.g., Dieffenfach and Dveksler, PCR Primer: A Laboratory Manual (1995)). Primers can be used, e.g., to amplify either the full length sequence or a probe of from one to several hundred nucleotides, which is then used to screen a mammalian library for full-length Gγ12 clones.

Nucleic acids encoding Gγ12 polypeptides can also be isolated from expression libraries using antibodies as probes. Such polyclonal or monoclonal antibodies can be raised using, for example, the sequence of SEQ ID NO: 1, or derivatives or fragments thereof. In addition, antibodies specific for mGγ12 can be obtained by further selecting the antibodies that bind to the sequence of SEQ ID NO: 1, but do not bind, for example, the sequence of SEQ ID NO: 4.

Polymorphic variants, alleles, and interspecies homologs that are substantially identical to a Gγ12 gene can be isolated using, for example, mGγ12 nucleic acid probes, and oligonucleotides under stringent hybridization conditions, by screening libraries. Alternatively, expression libraries can be used to clone Gγ12 polymorphic variants, alleles, and interspecies homologs, by detecting expressed homologs immunologically with antisera or purified antibodies made, for example, against an mGγ12 polypeptide, which also recognize and selectively bind to the mGγ12 homolog.

More distantly related Gγ12 homologs can be identified using any of a number of well known techniques, including by hybridizing a Gγ12 probe with a genomic or cDNA library using moderately stringent conditions, or under low stringency conditions. Also, a distant homolog can be amplified from a nucleic acid library using degenerate primer sets, i.e., primers that incorporate all possible codons encoding a given amino acid sequence, in particular based on a highly conserved amino acid stretch. Such primers are well known by those of skill, and numerous programs are available, e.g., on the Internet, for degenerate primer design.

To make a cDNA library, one should choose a source that is rich in Gγ12 mRNA, e.g., cells isolated from a large variety of tissues such as, e.g., liver, thymus, spleen, kidney, placenta, lung, kidney, pancreas, prostate, testis, ovary, small intestine, colon, etc. The mRNA is then made into cDNA using reverse transcriptase, ligated into a recombinant vector, and transfected into a recombinant host for propagation, screening and cloning. Methods for making and screening cDNA libraries are well known (see, e.g., Gubler and Hoffman (1983) Gene 25:263-269; Sambrook et al., supra; Ausubel et al., supra).

For a genomic library, the DNA is extracted from the tissue or cells and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton and Davis (1977) Science 196:180-182. Colony hybridization is carried out as generally described in Grunstein et al. (1975) Proc. Natl. Acad. Sci. USA., 72:3961-3965.

An alternative method of isolating a Gγ12 nucleic acid and its homologs combines the use of synthetic oligonucleotide primers and amplification of an RNA or DNA template (see, U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of Gγ12 genes directly from mRNA, from cDNA, from genomic libraries or cDNA libraries. Degenerate oligonucleotides can be designed to amplify Gγ12 homologs (for example mGγ12 homologs) using specific sequences (for example, the sequences described in SEQ ID NO: 2 or 3). Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of Gγ12-encoding mRNA in physiological samples, for nucleic acid sequencing, or for other purposes. Genes amplified by the PCR reaction can be purified from agarose gels and cloned into an appropriate vector.

Synthetic oligonucleotides can be used to construct recombinant Gγ12 genes for use as probes or for expression of protein. This method is performed using a series of overlapping oligonucleotides usually 40-120 bp in length, representing both the sense and non-sense strands of the gene. These DNA fragments are then annealed, ligated and cloned. Alternatively, amplification techniques can be used with precise primers to amplify a specific subsequence of the Gγ12 nucleic acid. The specific subsequence is then ligated into an expression vector.

The nucleic acid encoding a Gγ12 polypeptide is typically cloned into intermediate vectors before transformation into prokaryotic or eukaryotic cells for replication and/or expression. These intermediate vectors are typically prokaryote vectors, e.g., plasmids, or shuttle vectors. Vectors, cells, and transfection methods are well known to those of skill and are described, e.g., in Ausubel et al. or in Sambrook et al., both supra.

Optionally, nucleic acids will be used that encode chimeric proteins comprising a Gγ12 polypeptide or fragments thereof in combination with a heterologous polypeptide or polypeptides. Suitable heterologous proteins include, but are not limited to, e.g., luciferase, green fluorescent protein (GFP), and β-gal, each of which is well known in the art.

In certain embodiments, Gγ12 polynucleotides will be detected using hybridization-based methods to determine, e.g., Gγ12 RNA levels or to detect particular DNA sequences. For example, gene expression of Gγ12 can be analyzed by techniques known in the art, e.g., Northern blotting, reverse transcription and amplification of mRNA, dot blotting, in situ hybridization, RNase protection, probing DNA microchip arrays, and the like. In one embodiment, high density oligonucleotide analysis technology (e.g., GeneChip™) is used to identify homologs and polymorphic variants of Gγ12, or to monitor levels of Gγ12 mRNA. In the case where a homolog is linked to a known disease, they can be used with GeneChip™ as a diagnostic tool in detecting the disease in a biological sample (see, e.g., Gunthand et al. (1998) AIDS Res. Hum. Retroviruses 14: 869-876; Kozal et al. (1996) Nat. Med. 2:753-759; Matson et al. (1995) Anal. Biochem. 224:110-106; Lockhart et al. (1996) Nat. Biotechnol. 14:1675-1680; Gingeras et al. (1998) Genome Res. 8:435-448; Hacia et al. (1998) Nucleic Acids Res. 26:3865-3866).

Gγ12 DNA sequences, e.g., a particular allele, can be detected in a mammal using Southern blot hybridization, i.e., by isolating genomic DNA, performing a restriction digest on the isolated DNA, separating the restriction fragments electrophoretically, e.g., in an agarose gel, and transferring the separated DNA to a membrane and probing with a specific, labeled sequence. Southern blotting is well known to those of skill, and is taught in numerous sources, including Ausubel et al. and Sambrook et al., both supra.

In other embodiments, e.g., to detect tissue specific or temporal patterns of gene expression, a Gγ12 polynucleotide is detected using in situ hybridization. In in situ hybridization, the target nucleic acid is liberated from its cellular surroundings in such as to be available for hybridization within the cell while preserving the cellular morphology for subsequent interpretation and analysis. The following articles provide an overview of the art of in situ hybridization: Singer et al. (1986) Biotechniques 4:230-250; Haase et al., Methods in Virology, vol. VII, pp. 189-226 (1984); and Nucleic Acid Hybridization: A Practical Approach (Hames et al., eds. 1987).

C. Expression in Prokaryotes and Eukaryotes

Often, a cloned Gγ12 sequence will be expressed in a prokaryotic or eukaryotic cell to obtain expression, i.e., production of the encoded mRNA or protein. For example, in numerous embodiments, a Gγ12 polynucleotide will be introduced into a cell to modulate the level of Gγ12 activity in the cell, and thereby to modulate the level of estrogen signaling within cells of a patient. To obtain high level expression of a cloned gene or nucleic acid, such as a cDNA encoding a Gγ12 polypeptide, a Gγ12 sequence is typically subcloned into an expression vector that contains a strong promoter to direct transcription, a transcription/translation terminator, and, for a nucleic acid encoding a protein, a ribosome binding site for translational initiation. Suitable bacterial promoters are well known in the art and are described, e.g., in Sambrook et al. and Ausubel et al., both supra. Bacterial expression systems for expressing the Gγ12 protein are available in, e.g., E coli, Bacillus sp., and Salmonella (Palva et al. (1983) Gene 22:229-235; Mosbach et al. (1983) Nature 302:543-545). Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

For therapeutic applications, Gγ12 nucleic acids are introduced into a cell, in vitro, in vivo, or ex vivo, using any of a large number of methods including, but not limited to, infection with viral vectors, liposome-based methods, biolistic particle acceleration (the gene gun), and naked DNA injection. Such therapeutically useful nucleic acids include, but are not limited to, coding sequences for full-length Gγ12, coding sequences for a Gγ12 fragment, derivative, or variant, Gγ12 antisense sequences, and Gγ12 ribozymes. Typically, such sequences will be operably linked to a promoter, but in numerous applications a nucleic acid will be administered to a cell that is itself directly therapeutically effective, e.g., certain antisense or ribozyme molecules.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the Gγ12-encoding nucleic acid in host cells. A typical expression cassette thus contains a promoter operably linked to the nucleic acid sequence encoding a Gγ12 polypeptide, and signals required for efficient polyadenylation of the transcript, ribosome binding sites, and translation termination. The nucleic acid sequence encoding a Gγ12 polypeptide may be linked to a cleavable signal peptide sequence to promote secretion of the encoded protein by the transfected cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification, such as neomycin, thymidine kinase, hygromycin B phosphotransferase, and dihydrofolate reductase. Alternatively, high yield expression systems not involving gene amplification are also suitable, such as using a baculovirus vector in insect cells, with a sequence encoding a Gγ12 polypeptide under the direction of the polyhedrin promoter or other strong baculovirus promoters.

The elements that are typically included in expression vectors also include a replicon that functions in E coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of a Gγ12 protein, which are then purified using standard techniques (see, e.g., Colley et al. (1989) J. Biol. Chem. 264:17619-17622; Guide to Protein Purification, in Methods in Enzymology, vol. 182 (Deutscher, ed., 1990)). Transformation of eukaryotic and prokaryotic cells are performed according to standard techniques (see, e.g., Morrison (1977) J. Bact. 132:349-351; Clark-Curtiss and Curtiss, Methods in Enzymology 101:347-362 (Wu et al., eds; 1983)).

Any of the well known procedures for introducing foreign nucleotide sequences into host cells may be used to introduce the expression vector. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing a Gγ12 gene.

After the expression vector is introduced into the cells, the transfected cells are cultured under conditions favoring expression of the Gγ12 polypeptide, which is recovered from the culture using standard techniques identified below. Methods of culturing prokaryotic or eukaryotic cells are well known and are taught, e.g., in Ausubel et al., Sambrook et al., both supra, and in Freshney, Culture of Animal Cells, 3d. Ed., (1993), A Wiley-Liss Publication.

### IV. Purification of Gγ12 Polypeptides

Either naturally occurring or recombinant Gγ12 polypeptides can be purified for use in functional assays, binding assays, diagnostic assays, and other applications. Optionally, recombinant Gγ12 polypeptides are purified. Naturally occurring Gγ12 polypeptides are purified, e.g., from mammalian tissue such as, e.g., liver, thymus, spleen, kidney, placenta, lung, kidney, pancreas, prostate, testis, ovary, small intestine, colon, or any other source of a Gγ12 homolog. Recombinant Gγ12 polypeptides are purified from any suitable bacterial or eukaryotic expression system, e.g., CHO cells or insect cells. Here and throughout the specification, in some embodiments of the present invention, the Gγ12 polypeptides are mouse Gγ12 polypeptides (mGγ12).

Gγ12 proteins may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., both supra).

A number of procedures can be employed when recombinant Gγ12 polypeptide is being purified. For example, proteins having established molecular adhesion properties can be reversibly fused to the Gγ12 polypeptide. With the appropriate ligand, a Gγ12 polypeptide can be selectively adsorbed to a purification column and then freed from the column in a relatively pure form. The fused protein is then removed by enzymatic activity. Gγ12 proteins can also be purified using immunoaffinity columns.

A. Purification of Gγ12 Protein from Recombinant Cells

Recombinant proteins are expressed by transformed bacteria or eukaryotic cells such as CHO cells or insect cells in large amounts, typically after promoter induction, but expression can be constitutive. Promoter induction with IPTG is one example of an inducible promoter system. Cells are grown according to standard procedures in the art. Fresh or frozen cells are used for isolation of protein.

Proteins expressed in bacteria may form insoluble aggregates (“inclusion bodies”). Several protocols are suitable for purification of Gγ12 inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., and Ausubel et al., both supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing reformation of immunologically and/or biologically active protein. Other suitable buffers are known to those skilled in the art. Gγ12 polypeptides are separated from other bacterial proteins by standard separation techniques, e.g., with Ni-NTA agarose resin.

Alternatively, it is possible to purify Gγ12 polypeptides from bacteria periplasm. After lysis of the bacteria, when a Gγ12 protein is exported into the periplasm of the bacteria, the periplasmic fraction of the bacteria can be isolated by cold osmotic shock in addition to other methods known to those of skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying Gγ12 Polypeptides

1. Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

2. Size Differential Filtration

The molecular weight of a Gγ12 protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

Gγ12 proteins can also be separated from other proteins on the basis of their size, net surface charge, hydrophobicity, and affinity for heterologous molecules. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### V. Antibodies to Gγ12 Family Members

In numerous embodiments of the present invention, antibodies that specifically bind to Gγ12 polypeptides will be used. Such antibodies have numerous applications, including for the modulation of Gγ12 activity and for immunoassays to detect Gγ12, and variants, derivatives, fragments, etc. of Gγ12. Immunoassays can be used to qualitatively or quantitatively analyze a Gγ12 polypeptide. A general overview of the applicable technology can be found in Harlow and Lane, Antibodies: A Laboratory Manual (1988). In some embodiments, the antibodies of the present invention specifically bind to the mGγ12 and do not bind to Gγ12 proteins from a different species, such as, e.g., human Gγ12 .

A. Antibody Production

Methods of producing polyclonal and monoclonal antibodies that react specifically with Gγ12 polypeptides are known to those of skill in the art (see, e.g., Coligan, Current Protocols in Immunology (1991); Harlow and Lane, supra; Goding, Monoclonal Antibodies: Principles and Practice (2d ed. 1986); and Kohler and Milstein (1975) Nature 256:495-497). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al. (1989) Science 246:1275-1281; Ward et al. (1989) Nature 341:544-546).

A number of Gγ12 comprising immunogens may be used to produce antibodies specifically reactive with a Gγ12 polypeptide. For example, a recombinant Gγ12 protein, in particular an mGγ12 protein, or an antigenic fragment thereof, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the Gγ12 polypeptide. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler and Milstein (1976) Eur. J. Immunol. 6:511-519). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al. (1989) Science 246:1275-1281.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-Gγ12 proteins, or even related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Using Gγ12-specific antibodies, individual Gγ12 proteins can be detected by a variety of immunoassay methods. For a review of immunological and immunoassay procedures, see Basic and Clinical Immunology (Stites and Terr eds., 7th ed. 1991). Moreover, the immunoassays of the present invention can be performed in any of several configurations, which are reviewed extensively in Enzyme Immunoassay (Maggio, ed., 1980); and Harlow and Lane, supra.

B. Immunological Binding Assays

Gγ12 proteins can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites and Terr, eds., 7th ed. 1991). Immunological binding assays (or immunoassays) typically use an antibody that specifically binds to a protein or antigen of choice (for example, an mGγ12 protein or an antigenic subsequence thereof). The antibody (e.g., the anti-mGγ12 antibody) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled Gγ12 polypeptide (e.g., a labeled mGγ12 polypeptide) or a labeled anti-Gγ12 antibody (e.g., a labeled anti-mGγ12 antibody). Alternatively, the labeling agent may be a third moiety, such a secondary antibody, that specifically binds to the antibody/Gγ12 complex (a secondary antibody is typically specific to antibodies of the species from which the first antibody is derived). Other proteins capable of specifically binding immunoglobulin constant regions, such as protein A or protein G, may also be used as the label agent. These proteins exhibit a strong nonimmunogenic reactivity with immunoglobulin constant regions from a variety of species (see, e.g., Kronval et al. (1973) J. Immunol. 111:1401-1406; Akerstrom et al. (1985) J. Immunol. 135:2589-2542). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

1. Noncompetitive Assay Formats

Immunoassays for detecting a Gγ12 protein in a sample may be either competitive or noncompetitive. Noncompetitive immunoassays are assays in which the amount of antigen is directly measured. In one preferred “sandwich” assay, for example, the anti-Gγ12 antibodies can be bound directly to a solid substrate on which they are immobilized. These immobilized antibodies then capture the Gγ12 protein present in the test sample. The Gγ12 protein is thus immobilized and is then bound by a labeling agent, such as a second Gγ12 antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

2. Competitive Assay Formats

In competitive assays, the amount of Gγ12 protein present in the sample is measured indirectly by measuring the amount of a known, added (exogenous) Gγ12 protein displaced (competed away) from an anti-Gγ12 antibody by the unknown Gγ12 protein present in a sample. In one competitive assay, a known amount of Gγ12 protein is added to a sample and the sample is then contacted with an antibody that specifically binds to the Gγ12 protein. The amount of exogenous Gγ12 protein bound to the antibody is inversely proportional to the concentration of Gγ12 protein present in the sample. In a particularly preferred embodiment, the antibody is immobilized on a solid substrate. The amount of Gγ12 protein bound to the antibody may be determined either by measuring the amount of Gγ12 protein present in a Gγ12/antibody complex, or alternatively by measuring the amount of remaining uncomplexed protein. The amount of Gγ12 protein may be detected by providing a labeled Gγ12 molecule.

A hapten inhibition assay is another preferred competitive assay. In this assay the known Gγ12 protein is immobilized on a solid substrate. A known amount of anti-Gγ12 antibody is added to the sample, and the sample is then contacted with the immobilized Gγ12. The amount of anti-Gγ12 antibody bound to the known immobilized Gγ12 protein is inversely proportional to the amount of Gγ12 protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

3. Cross-Reactivity Determinations

Immunoassays in the competitive binding format can also be used for crossreactivity determinations. For example, a protein at least partially encoded by a nucleic acid having the sequence of SEQ ID NO: 2 or 3 can be immobilized on a solid support. Proteins (e.g., mGγ12 proteins and homologs) are added to the assay that compete for binding of the antisera to the immobilized antigen. The ability of the added proteins to compete for binding of the antisera to the immobilized protein is compared to the ability of the mGγ12 polypeptide encoded by nucleic acid having the sequence of SEQ ID NO: 2 or 3 to compete with itself The percent cross-reactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a Gγ12 protein, to the immunogen protein (e.g., the mGγ12 protein encoded by nucleic acid having the sequence of SEQ ID NO: 2 or 3). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the immunogen protein of interest (e.g., the protein encoded by SEQ ID NO: 2 or 3) that is required to inhibit 50% of binding, then the second protein is said to specifically bind to the polyclonal antibodies generated to a Gγ12 immunogen (e.g., a mGγ12 immunogen).

Polyclonal antibodies that specifically bind to a Gγ12 protein from a particular species can be made by subtracting out cross-reactive antibodies using Gγ12 homologs. For example, antibodies specific to mGγ12 (SEQ ID NO: 1) can be made by subtracting out antibodies that are cross-reactive with, e.g., a human Gγ12 having the sequence of SEQ ID NO: 4 or a rat Gγ12 (GenBank acc: AF022091). In an analogous fashion, antibodies specific to a particular GTP-binding protein gamma-12 protein can be obtained in an organism with multiple GTP-binding protein gamma-12 genes.

4. Other Assay Formats

Western blot (immunoblot) analysis is used to detect and quantify the presence of Gγ12 protein in a sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the Gγ12 protein. The anti-Gγ12 polypeptide antibodies specifically bind to the Gγ12 polypeptide on the solid support. These antibodies may be directly labeled or alternatively may be subsequently detected using labeled antibodies (e.g., labeled sheep anti-mouse antibodies) that specifically bind to the anti-Gγ12 antibodies.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

5. Reduction of Nonspecific Binding

One of skill in the art will appreciate that it is often desirable to minimize nonspecific binding in immunoassays. Particularly, where the assay involves an antigen or antibody immobilized on a solid substrate it is desirable to minimize the amount of nonspecific binding to the substrate. Means of reducing such nonspecific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

6. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵s, ¹⁴C, or ³²p), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Nonradioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize a Gγ12 protein, or secondary antibodies that recognize anti-Gγ12.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, e.g., U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### VI. Modulating Gγ12 Activity in Cells

A. Assays for Modulators of Gγ12 Proteins

In numerous embodiments of this invention, the level of Gγ12 activity will be modulated in a cell by administering to the cell, in vivo or in vitro, any of a large number of Gγ12-modulating molecules, e.g., polypeptides, antibodies, amino acids, nucleotides, lipids, carbohydrates, or any organic or inorganic molecule. Such Gγ12 modulators are particularly useful in the treatment of any of a number of diseases, such as, e.g., atherosclerosis, osteoporosis, Alzheimer's disease, Parkinson's disease, breast cancer, etc.

To identify molecules capable of modulating Gγ12, assays will be performed to detect the effect of various compounds on Gγ12 activity in a cell. Such assays can involve the identification of compounds that interact with Gγ12 proteins, either physically or genetically, and can thus rely on any of a number of standard methods to detect physical or genetic interactions between compounds. Such assays can also involve the identification of compounds that affect Gγ12 expression, activity or other properties, such as its phosphorylation or ability to bind other proteins. Such assays can also involve the detection of Gγ12 activity in a cell, either in vitro or in vivo. Such cell-based assays can be performed in any type of cell, e.g., a cell that naturally expresses Gγ12, or a cultured cell that produces Gγ12 due to recombinant expression.

B. Assays for Gγ12-Interacting Compounds

In certain embodiments, assays will be performed to identify molecules that physically or genetically interact with Gγ12 proteins. Such molecules can be any type of molecule, including polypeptides, polynucleotides, amino acids, nucleotides, carbohydrates, lipids, or any other organic or inorganic molecule. Such molecules may represent molecules that normally interact with Gγ12 proteins, for example, to effect estrogen signaling, or may be synthetic or other molecules that are capable of interacting with Gγ12 proteins and that can potentially be used to modulate Gγ12 proteins activity in cells, or used as lead compounds to identify classes of molecules that can interact with and/or modulate Gγ12 proteins. Such assays may represent physical binding assays, such as affinity chromatography, immunoprecipitation, two-hybrid screens, or other binding assays, or may represent genetic assays as described infra.

In any of the binding or functional assays described herein, in vivo or in vitro, any Gγ12 protein, or any derivative, variation, homolog, or fragment of a Gγ12 protein, can be used. Preferably, the Gγ12 protein is at least about 80% identical to SEQ ID NO: 1. Alternatively, a fragment of a Gγ12 protein can be used. Such fragments can be used alone, in combination with other Gγ12 fragments, or in combination with sequences from heterologous proteins, e.g., the fragments can be fused to a heterologous polypeptide, thereby forming a chimeric polypeptide.

Compounds that interact with Gγ12 proteins can be isolated based on an ability to specifically bind to a Gγ12 protein or fragment thereof. In numerous embodiments, the Gγ12 protein or protein fragment will be attached to a solid support. In one embodiment, affinity columns are made using the Gγ12 polypeptide, and physically-interacting molecules are identified. It will be apparent to one of skill that chromatographic techniques can be performed at any scale and using equipment from many different manufacturers (e.g., Pharmacia Biotech). In addition, molecules that interact with Gγ12 proteins in vivo can be identified by co-immunoprecipitation or other methods, i.e., immunoprecipitating Gγ12 proteins using anti-Gγ12 antibodies from a cell or cell extract, and identifying compounds, e.g., proteins, that are precipitated along with the Gγ12 protein. Such methods are well known to those of skill in the art and are taught, e.g., in Ausubel et al., Sambrook et al., Harlow and Lane, all supra.

Two-hybrid screens can also be used to identify polypeptides that interact in vivo with a Gγ12 polypeptide or a fragment thereof (Fields et al. (1989) Nature 340:245-246). Such screens comprise two discrete, modular domains of a transcription factor protein, e.g., a DNA binding domain and a transcriptional activation domain, which are produced in a cell as two separate polypeptides, each of which also comprises one of two potentially binding polypeptides. If the two potentially binding polypeptides in fact interact in vivo, then the DNA binding and the transcriptional activating domain of the transcription factor are united, thereby producing expression of a target gene in the cell. The target gene typically encodes an easily detectable gene product, e.g., β-galactosidase, GFP, or luciferase, which can be detected using standard methods. In the present invention, a Gγ12 polypeptide is fused to one of the two domains of the transcription factor, and the potential Gγ12-binding polypeptides (e.g., encoded by a cDNA library) are fused to the other domain. Such methods are well known to those of skill in the art, and are taught, e.g., in Ausubel et al., supra.

C. Assays for Gγ12 Protein Activity

Gγ12 genes and their alleles and polymorphic variants encode GTP-binding protein gamma-12 subunits. Accordingly, the activity of Gγ12 polypeptides can be assessed using a variety of in vitro and in vivo assays to determine functional, chemical, and physical effects, e.g., directly measuring the activity of Gγ12. The activity of Gγ12 can be measured by, for example, measuring the activity of Gγ12, of the γ/β complex, or of the GTP-binding protein, by, e.g., measuring the expression or activity of downstream effectors (such as, e.g., adenylate cyclase, K+ channels, Ca2+ channels, phospholipase A2, phospholipase c-β, phosphatidylinositol 3-kinase, etc.), measuring the binding of Gγ12 to heterologous proteins (e.g., GTP-binding protein alpha or beta subunits) or to other molecules (e.g., radioactive binding), measuring Gγ12 protein and/or RNA levels, or measuring other aspects of Gγ12 polypeptides (e.g., phosphorylation levels, transcription levels, and the like). Such assays can be used to test for both activators and inhibitors of Gγ12 proteins. Modulators can also be genetically altered versions of Gγ12 proteins, e.g., dominant negative forms of Gγ12 or of proteins that interact with Gγ12 (e.g., GTP-binding protein alpha or beta subunits). Such modulators of activity are useful for, e.g., many diagnostic and therapeutic applications.

In some embodiments, the Gγ12 protein of the assay will be a recombinant or naturally occurring mGγ12 polypeptide having the sequence of SEQ ID NO: 1 or conservatively modified variants thereof. Alternatively, the Gγ12 protein of the assay will be derived from another eukaryote and include an amino acid subsequence having amino acid sequence identity to SEQ ID NO: 1. Generally, the amino acid sequence identity will be at least 80%, optionally at least 85%-95%, or more. Optionally, the polypeptide of the assays will comprise a fragment of a Gγ12 protein. In certain embodiments, a fragment of a Gγ12 protein is fused to a heterologous polypeptide, thereby forming a chimeric polypeptide. Such chimeric polypeptides are also useful, e.g., in assays to identify modulators of Gγ12.

Samples or assays that are treated with a potential Gγ12 protein inhibitor or activator are compared to control samples without the test compound, to examine the extent of modulation. Control samples (untreated with activators or inhibitors) are assigned a relative Gγ12 activity value of 100. Inhibition of a Gγ12 protein is achieved when the Gγ12 activity value relative to the control is about 90%, optionally 50%, optionally 25-0%. Activation of a Gγ12 protein is achieved when the Gγ12 activity value relative to the control is 110%, optionally 150%, 200-500%, or 1000-2000%.

The effects of the test compounds upon the function of the polypeptides can be measured by examining any of the parameters described above. Any suitable physiological change that affects Gγ12 activity can be used to assess the influence of a test compound on the polypeptides of this invention. When the functional consequences are determined using intact cells or animals, one can also measure a variety of effects such as, for example, changes in estrogen signaling and/or cardiovascular protection, as indicated by, e.g., changes in the activity or expression of vasoactive molecules.

D. Identifying Estrogen Receptor Agonists and Antagonists

In a preferred embodiment, transcription levels of Gγ12 are measured to assess the effects of a test compound on estrogen signaling, e.g., mediated by an estrogen receptor α. A host cell containing an estrogen-receptor of interest is contacted with a test compound for a sufficient amount of time to effect any interactions, and the level of Gγ12 expression is then measured. The amount of time to effect such interactions may be empirically determined, such as by running a time course and measuring the level of transcription as a function of time.

Such assays can be performed using any suitable eukaryotic cells, including, e.g., mammalian cells, insect cells, plant or yeast cells using standard methods. A cell type will be selected that naturally expresses an estrogen receptor, e.g., an estrogen receptor α, or which is induced to express an estrogen receptor by, for example, introducing a heterologous polynucleotide encoding the receptor, operably linked to a promoter, into the cell.

The amount of transcription may be measured using any method known to those of skill in the art to be suitable. For example, mRNA expression of the protein of interest (e.g., mGγ12) may be detected using Northern blots, reverse transcriptase-polymerase chain reaction (RT-PCR), or any other standard method, using, e.g., probes or primers designed from a specific nucleotide sequence encoding a Gγ12 polypeptide (e.g., SEQ ID NO: 2 or 3). Alternatively, the expression of the Gγ12 can be detected by detecting the level of the polypeptide products using immunoassays or other assays to detect Gγ12 activity. Such assays can use natural forms of Gγ12 or can use a Gγ12 fusion, e.g., Gγ12 transcript fused to a reporter sequence, e.g., a sequence encoding chloramphenicol acetyltransferase, luciferase, β-galactosidase, GFP, or alkaline phosphatase. Alternatively, one of these marker sequences can be operably linked to an mGγ12 promoter (see, e.g., Mistili and Spector (1997) Nature Biotechnology 15:961-964). Furthermore, any polynucleotide typically expressed following Gγ12 activation, e.g., adenylate cyclase, K+ channels, Ca2+ channels, phospholipase A2, phospholipase c-β, or phosphatidylinositol 3-kinase, can be used.

The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

A compound that causes an increase in the amount of Gγ12 expression, as detected by any of the herein-described methods, is a candidate for an estrogen receptor agonist that would be useful, e.g., in the inhibition of osteoporosis, atherosclerosis, Alzheimer's Disease, or Parkinson's Disease, and a compound that causes a decrease in the amount of Gγ12 expression is a candidate for an estrogen-receptor antagonist, which would be useful in the inhibition of, e.g., breast cancer. Candidate agonists or antagonists can be further characterized by any of a number of methods, including, e.g., directly examining their interactions with estrogen receptors, examining their ability to alter Gγ12 expression in cells that do not express estrogen receptors, examining the estrogen receptor specificity of the candidate (i.e., estrogen receptor α or β), etc.

In preferred embodiments, the detection of the level of Gγ12 expression in the presence or absence of the test compound is tested simultaneously for a large number of test compounds, e.g., using high throughput screening.

E. Modulators and Binding Compounds

The compounds tested as modulators of a Gγ12 protein can be any small chemical compound, or a biological entity, such as a protein, sugar, nucleic acid or lipid. Alternatively, modulators can be genetically altered versions of a Gγ12 gene. Typically, test compounds will be small chemical molecules and peptides. Essentially any chemical compound can be used as a potential modulator or binding compound in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as, e.g., a polypeptide library is formed by combining a set of chemical building blocks (e.g., amino acids) in every possible way for a given compound length (e.g., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka (1991) Int. J. Pept. Prot. Res. 37:487-493; and Houghton et al. (1991) Nature 354:84-88). Other chemistries for generating chemical diversity libraries can also be used. Such chemistries include, but are not limited to, peptoids (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (Hobbs et al. (1993) Proc. Nat. Acad. Sci. USA 90:6909-6913), vinylogous polypeptides (Hagihara et al. (1992) J. Amer. Chem. Soc. 114:6568), nonpeptidal peptidomimetics with glucose scaffolding (Hirschmann et al. (1992) J. Amer. Chem. Soc. 114:9217-9218), analogous organic syntheses of small compound libraries (Chen et al (1994) J. Amer. Chem. Soc. 116:2661), oligocarbamates (Cho et al (1993) Science 261:1303), and/or peptidyl phosphonates (Campbell et al. (1994) J. Org. Chem. 59:658), nucleic acid libraries (see Ausubel et al.; Berger et al.; and Sambrook et al., all supra), peptide nucleic acid libraries (see, e.g., U.S. Pat. No. 5,539,083), antibody libraries (see, e.g., Vaughn et al., (1996) Nature Biotechnology, 14(3):309-314 and PCT/US96/10287), carbohydrate libraries (see, e.g., Liang et al. (1996) Science, 274:1520-1522; and U.S. Pat. No. 5,593,853), small organic molecule libraries (e.g., benzodiazepines, see, Baum CandEN, January 18, page 33 (1993); isoprenoids, see, U.S. Pat. No. 5,569,588; thiazolidinones and metathiazanones, see, U.S. Pat. No. 5,549,974; pyrrolidines, see, U.S. Pat. Nos. 5,525,735 and 5,519,134; morpholino compounds, see, U.S. Pat. No. 5,506,337; benzodiazepines, see, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

1. Solid State and Soluble High Throughput Assays

In one embodiment, the invention provides soluble assays using molecules such as an N-terminal or C-terminal fragment either alone or covalently linked to a heterologous protein to create a chimeric molecule. In another embodiment, the invention provides solid phase based in vitro assays in a high throughput format, where a Gγ12 fragment, Gγ12 chimeric molecule, Gγ12 protein, or cell or tissue expressing a Gγ12 protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Alabama. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield (1963) J. Am. Chem. Soc. 85:2149-2154 (describing solid phase synthesis of, e.g., peptides); Geysen et al. (1987) J. Immun. Meth. 102:259-274 (describing synthesis of solid phase components on pins); Frank and Doring (1988) Tetrahedron 44:60316040 (describing synthesis of various peptide sequences on cellulose disks); Fodor et al. (1991) Science, 251:767-777; Sheldon et al. (1993) Clinical Chemistry 39(4):718-719; and Kozal et al. (1996) Nature Medicine 2(7):753759 (all describing arrays of biopolymers fixed to solid substrates)). Nonchemical approaches for fixing tag binders to substrates include other common methods, such as heat, cross-linking by UV radiation, and the like.

2. Computer-Based Assays

Yet another assay for compounds that modulate Gγ12 protein activity involves computer assisted drug design, in which a computer system is used to generate a three-dimensional structure of a Gγ12 protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a Gγ12 polypeptide into the computer system. In one embodiment, the nucleotide sequence encoding the polypeptide comprises SEQ ID NO: 2 or 3, and conservatively modified versions thereof. In another embodiment, the amino acid sequence, comprising SEQ ID NO: 1, or conservatively modified versions thereof, represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the Gγ12 protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of Gγ12 genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated Gγ12 genes (e.g., the mutated mGγ12 gene) involves receiving input of a first nucleic acid sequence (e.g., the nucleic acid sequence of SEQ ID NO: 2 or 3) or a first amino acid sequence (e.g., the amino acid sequence of SEQ ID NO: 1) and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various Gγ12 genes, and mutations associated with disease states and genetic traits.

### VII. Modulating Gγ12 Activity/Expression to Treat Diseases or Conditions

In numerous embodiments of this invention, a compound, e.g., nucleic acid, polypeptide, or other molecule is administered to a patient, in vivo or ex vivo, to effect a change in Gγ12 activity or expression in the patient. Such compounds can be nucleic acids encoding full length Gγ12 polypeptides, e.g., as shown as SEQ ID NO: 1, or any derivative, fragment, or variant thereof, operably linked to a promoter. Suitable nucleic acids also include inhibitory sequences such as antisense or ribozyme sequences, which can be delivered in, e.g., an expression vector operably linked to a promoter, or can be delivered directly. Also, any nucleic acid that encodes a polypeptide that modulates the expression of Gγ12 can be used. In general, nucleic acids can be delivered to cells using any of a large number of vectors or methods, e.g., retroviral, adenoviral, or adeno-associated virus vectors, liposomal formulations, naked DNA injection, and others. All of these methods are well known to those of skill in the art.

Proteins can also be delivered to a patient to modulate Gγ12 activity. In preferred embodiments, a polyclonal or monoclonal antibody that specifically binds to Gγ12 will be delivered. In addition, any polypeptide that interacts with and/or modulates Gγ12 activity can be used, e.g., a polypeptide that is identified using the presently described assays, or any dominant negative form of Gγ12 or a Gγ12-interacting protein, e.g., GTP-binding protein β or α subunits, etc. In addition, polypeptides that affect Gγ12 expression can be used.

Further, any compound that is found to or designed to interact with and/or modulate the activity of Gγ12 can be used. For example, any compound that is found, using the methods described herein, to bind to or modulate the activity of Gγ12 can be used.

Any of the above-described molecules can be used to increase or decrease the expression or activity of Gγ12 , or to otherwise affect the properties and/or behavior of Gγ12 polypeptides or polynucleotides, e.g., stability, phosphorylation, kinase activity, interactions with other proteins, etc. The present compounds can thus be used to treat any of a number of diseases, including, but not limited to (a) pulmonary diseases and diseases of the airway, including but not limited to, e.g., cardiovascular diseases including but not limited to stroke and atherosclerosis, osteoporosis, breast cancer, Alzheimer's disease, Parkinson's disease, etc.

Administration of any of the present molecules can be achieved by any of the routes normally used for introducing a modulator compound into ultimate contact with the tissue to be treated. The modulators are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The Gγ12 modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and nonaqueous solutions, isotonic sterile solutions, which can contain antioxidants, buffers, bacteriostats, and solutes that render the formulation isotonic, and aqueous and nonaqueous sterile suspensions that can include suspending agents, solubilizers, thickening agents, stabilizers, and preservatives. In the practice of this invention, compositions can be administered, for example, orally, nasally, topically, intravenously, intraperitoneally, intravesically or intrathecally. The formulations of compounds can be presented in unit-dose or multi-dose sealed containers, such as ampules and vials. Solutions and suspensions can be prepared from sterile powders, granules, and tablets of the kind previously described. The modulators can also be administered as part a of prepared food or drug.

The dose administered to a patient, in the context of the present invention should be sufficient to effect a beneficial response in the subject over time. The dose will be determined by the efficacy of the particular modulators employed and the condition of the subject, as well as the body weight or surface area of the area to be treated. The size of the dose also will be determined by the existence, nature, and extent of any adverse side-effects that accompany the administration of a particular compound or vector in a particular subject.

In determining the effective amount of the modulator to be administered, a physician may evaluate circulating plasma levels of the modulator, modulator toxicities, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the compound at various concentrations, as applied to the mass and overall health of the subject. Administration can be accomplished via single or divided doses.

### VIII. Transgenic Animals

Transgenic and chimeric non-human mammals and methods for generating them are described below. The mammals are useful, inter alia, for testing the function of Gγ12 in vivo, to generate models for the study of, e.g., atherosclerosis, osteoporosis, Alzheimer's disease, Parkinson's disease and breast cancer, and for the development of potential treatments for Gγ12 related, e.g., cardiovascular diseases and conditions.

Transgenic and chimeric non-human mammals are generated that contain cells that lack at least one functional endogenous allele for Gγ12. A “chimeric animal” includes some cells that lack the functional Gγ12 gene of interest and other cells that do not have the inactivated gene. A “transgenic animal,” in contrast, is made up of cells that have all incorporated the specific modification which renders the Gγ12 gene inactive or otherwise altered. While a transgenic animal is typically always capable of transmitting the mutant Gγ12 gene to its progeny, the ability of a chimeric animal to transmit the mutation depends upon whether the inactivated gene is present in the animal's germ cells. The modifications that inactivate or otherwise alter the Gγ12 gene can include, for example, insertions, deletions, or substitutions of one or more nucleotides. The modifications can interfere with transcription of the gene itself, with translation and/or stability of the resulting mRNA, or can cause the gene to encode an inactive or otherwise altered Gγ12 polypeptide, e.g., a Gγ12 polypeptide with modified binding properties or kinase activity.

The claimed methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, Ed., Genetic Engineering of animals, VCH Publ. (1993); Murphy and Carter, Eds., Transgenesis Techniques: Principles and Protocols (Methods in Molecular Biology, Vol. 18), (1993); and Pinkert, Ed., Transgenic Animal Technology: A Laboratory Handbook, Academic Press (1994).

In preferred embodiments, transgenic mice will be produced as described in Thomas et al. (1999) Immunol. 163:978-84; Kanakaraj et al. (1998) J. Exp. Med. 187:2073-9; or Yeh et al. (1997) Immunity 7:715-725.

Typically, a modified Gγ12 gene (e.g., a modified mGγ12 gene) is introduced, e.g., by homologous recombination, into embryonic stem cells (ES), which are obtained from preimplantation embryos and cultured in vitro (see, e.g., Hooper, Embryonal Stem Cells: Introducing Planned Changes into the Animal Germline (Modern Genetics, v. 1), Int'l. Pub. Distrib., Inc. (1993); Bradley et al. (1984) Nature 309:255-258). Subsequently, the transformed ES cell is combined with a blastocyst from a non-human animal, e.g., a mouse. The ES cells colonize the embryo and in some embryos form the germ line of the resulting chimeric animal (see, Jaenisch (1988) Science 240:1468-1474). Alternatively, ES cells or somatic cells that can reconstitute an organism (“somatic repopulating cells”) can be used as a source of nuclei for transplantation into an enucleated fertilized oocyte giving rise to a transgenic mammal (see, e.g., Wilmut et al. (1997) Nature 385:810-813).

Other methods for obtaining a transgenic or chimeric animal having a mutant Gγ12 gene in its genome is to contact fertilized oocytes with a vector that includes a polynucleotide that encodes a modified, e.g., inactive, Gγ12 polypeptide. In some animals, such as mice, fertilization is typically performed in vivo and fertilized ova are surgically removed. In other animals, particularly bovines, it is preferably to remove ova from live or slaughterhouse animals and fertilize the ova in vitro (See, WO 91/08216). In vitro fertilization permits the modifications to be introduced into substantially synchronous cells.

Fertilized oocytes are typically cultured in vitro until a pre-implantation embryo is obtained containing about 16-150 cells. The 16-32 cell stage of an embryo is described as a morula, whereas pre-implantation embryos containing more than 32 cells are termed blastocysts. These embryos show the development of a blastocoel cavity, typically at the 64 cell stage. The presence of a desired Gγ12 mutation in the cells of the embryo can be detected by methods known to those of skill in the art, e.g., Southern blotting, PCR, DNA sequencing, or other standard methods. Methods for culturing fertilized oocytes to the pre-implantation stage are described, e.g., by Gordon et al. (1984) Methods Enzymol. 101:414; Hogan et al. Manipulation of the Mouse Embryo: A Laboratory Manual, C.S.H.L. N.Y. (1986) (mouse embryo); Hammer et al. (1985) Nature 315:680 (rabbit and porcine embryos); Gandolfi et al. (1987) J. Reprod. Fert. 81:23-28; Reprod et al. (1988) J. Anim. Sci. 66:947-953 (ovine embryos) and Eyestone et al. (1989) J. Reprod. Fert. 85:715-720; Camous et al. (1984) J. Reprod. Fert. 72:779-785; and Heyman et al. (1987) Theriogenology 27:5968 (bovine embryos). Pre-implantation embryos may also be stored frozen for a period pending implantation.

Pre-implantation embryos are transferred to an appropriate female resulting in the birth of a transgenic or chimeric animal, depending upon the stage of development when the transgene is integrated. Chimeric mammals can be bred to form true germline transgenic animals. Chimeric mice and germline transgenic mice can also be ordered from commercial sources (e.g., Deltagen, San Carlos, Calif.).

Other methods for introducing mutations into mammalian cells or animals include recombinase systems, which can be employed to delete all or a portion of a locus of interest. Examples of recombinase systems include, the cre/lox system of bacteriophage P1 (see, e.g., Gu et al. (1994) Science 265:103-106; Terry et al. (1997) Transgenic Res. 6:349-356) and the FLP/FRT site specific integration system (see, e.g., Dymecki (1996) Proc. Natl. Acad. Sci. USA 93:6191-6196). In these systems, sites recognized by the particular recombinase are typically introduced into the genome at a position flanking the portion of the gene that is to be deleted. Introduction of the recombinase into the cells then catalyzes recombination which deletes from the genome the polynucleotide sequence that is flanked by the recombination sites. If desired, one can obtain animals in which only certain cell types lack the Gγ12 gene of interest, e.g., by using a tissue specific promoter to drive the expression of the recombinase (see, e.g., Tsien et al. (1996) Cell 87:1317-26; Brocard et al. (1996) Proc. Natl. Acad. Sci. USA 93:10887-10890; Wang et al. (1996) Proc. Natl. Acad. Sci. USA 93:3932-6; Meyers et al. (1998) Nat. Genet. 18: 136-41).

The presence of any mutation in a Gγ12 gene in a cell or animal can be detected using any method described herein, e.g., Southern blot, PCR, or DNA sequencing (see, e.g., Ausubel et al., supra).

### IX. Kits

Gγ12 genes and their homologs are useful tools for a number of applications, including, but not limited to, identifying estrogen-responsive cells for treating any of a large number of estrogen-associated diseases, such as, e.g., atherosclerosis, osteoporosis, breast cancer, Alzheimer's disease, Parkinson's disease, etc. Gγ12 specific reagents that specifically hybridize to Gγ12 nucleic acids, such as Gγ12 probes and primers, and Gγ12 specific reagents that specifically bind to or modulate the activity of a Gγ12 protein, e.g., anti-Gγ12 antibodies or other compounds, can thus be provided in a kit for the practice of any of the applications described herein.

Nucleic acid assays for the presence of DNA and RNA for a Gγ12 polynucleotide in a sample include numerous techniques known to those skilled in the art, such as Southern analysis, Northern analysis, dot blots, RNase protection, S 1 analysis, amplification techniques such as PCR and LCR, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al. (1986) Biotechniques 4:230-250; Haase et al., Methods in Virology, vol. VII, pp. 189-226 (1984); and Nucleic Acid Hybridization: A Practical Approach (Hames et al., eds. 1987). In addition, an Gγ12 protein can be detected with the various immunoassay techniques described above. The test sample is typically compared to both a positive control (e.g., a sample expressing a recombinant Gγ12 protein) and a negative control.

The present invention also provides kits for screening for modulators of Gγ12 proteins or nucleic acids. Such kits can be prepared from readily available materials and reagents. For example, such kits can comprise any one or more of the following materials: Gγ12 nucleic acids or proteins, reaction tubes, and instructions for testing Gγ12 activity. Optionally, the kit contains a biologically active Gγ12 protein. A wide variety of kits and components can be prepared according to the present invention, depending upon the intended user of the kit and the particular needs of the user.

### X. Example

Expressed sequence tags (EST) that represent messenger RNA (mRNA) molecules that are more abundant in the liver of normal male mice treated with estrogen than in the liver of estrogen receptor alpha knockout (ERKO) male mice also treated with estrogen were obtained by subtraction cloning. The DNA sequence of complementary DNA (cDNA) clones representing the full-length mRNA molecules corresponding to one of the above mentioned EST's was obtained.

Two variants of the cDNA, designated variant 1 and variant 2, were obtained and the DNA sequences of these are shown shown as SEQ ID NO: 2 and 3, respectively. The sequences of variant 1 and variant 2 are identical except that variant 1 lacks a region of 53 bp present between nucleotides 87 and 139 of the sequence of variant 2 shown SEQ ID NO: 3. In addition, the first 56 nucleotides of variant 1 and the first 31 nucleotides of variant 2 are different from each other.

Variant 1 and variant 2 encode the same predicted protein of 72 amino acids that is shown in SEQ ID NO: 1. This protein initiates at an ATG codon (AUG in mRNA, encoding the amino acid methionine) at nucleotide 141 of the sequence of variant 1 shown in SEQ ID NO: 2 and nucleotide 169 of the sequence of variant 2 shown in SEQ ID NO: 3. This ATG codon lies downstream from the above-mentioned 53 bp exon such that the entire DNA sequence encoding this 72 amino acid protein lies within the region where the two variants are identical. The initiating ATG codon lies within a sequence that conforms to the consensus sequence for translation initiation as defined by Kozak (1989) J. Cell Biol. 108:229-241. For most mRNAs, translation initiates at the first AUG codon at the 5′ end of the mRNA. In the case of the cDNA sequences reported here there is a single ATG codon upstream of the ATG codon that initiates the 72 amino acid protein described above. This additional upstream ATG codon lies within the same sequence context in both variants and matches the Kozak consensus sequence for translation initiation. In both variants, these upstream ATG codons are however followed after 5 or 6 codons by in-frame stop codons. The ribosome may thus initiate transcription at the first AUG, terminate at the in-frame stop codon, scan down the mRNA until it reaches the second AUG codon and re-initiate translation. Alternatively, upstream AUG codons may lie within introns as has been shown for some genes, in which case the cDNA may represent a partially processed transcript in which this intron was not removed.

Searches of the public databases (GenBank, Swissprot) revealed that the cDNA sequences reported here have significant sequence similarity to members of the GTP-binding protein gamma subunit family of genes. Furthermore, the predicted 72 amino acid protein encoded by the cDNA sequences reported here exhibits a high degree of sequence similarity to members of the GTP-binding protein gamma subunit family from many species. Multiple alignments of these proteins indicated that the predicted 72 amino acid protein reported here is most similar to the human GTP-binding protein gamma-12 subunit shown as SEQ ID NO: 4 (70 out of 72 amino acids are identical). The cDNA sequence reported here and the predicted 72 amino acid protein encoded by it was designated as mouse GTP-binding protein gamma 12 subunit (mGγ12).

To date at least eleven mammalian G-protein γ subunits have been identified. The sequences of these γ subunits are more divergent than those of the α and β subunit families, suggesting a greater degree of functional divergence. Western blots with an antibody against the bovine G-protein γ-12 demonstrated expression of the rat γ-12 subunit in all of the 19 tissues examined (Morishata et al. (1995) J. Biol. Chem. 270:29469-29475). This suggested that, in contrast to the other G-protein γ subunits, the γ-12 subunit is expressed ubiquitously, at least in the rat. The primary G-protein gamma subunit in cultured rat aortic smooth muscle cells was shown to be γ-12, with only a small amount of γ-5, and no detectable γ-2, -3, or -7 (Morishata et al., supra). Furthermore, the γ-12 subunit was phosphorylated on Ser1 or Ser2 by protein kinase C, and this phosphorylation was observed in cultured aortic smooth muscle cells in response to the agonists angiotensin II and vasopressin, both of which are vasoconstrictors in vivo. Phosphorylation of the γ-12 subunit can modulate its ability to activate downstream effectors. Yasuda et al. ((1998) J. Biol. Chem. 273:21958-21965) showed that phosphorylation of the γ-12 subunit increased the efficiency of activation of adenylate cyclase but had no effect on its ability to activate phospholipase C-β.

Northern blot analysis was conducted with RNA prepared from the livers of normal male mice treated with estrogen, ERKO male mice treated with estrogen, and un-treated male mice using a hybridization probe derived from the mGγ12 cDNA. This experiment demonstrated that expression of the mGγ12 mRNA required the presence of a functional estrogen receptor alpha, and indicated that transcription of the mGγ12 gene in the liver was activated by estrogen specifically via the alpha but not the beta-receptor. This provided the first demonstration of estrogen receptor sub-type specific modulation of gene expression.

Both forms of the estrogen receptor (α and β) are expressed in the coronary artery and in cultured aortic smooth muscle cells of primates (Register and Adams (1998) J. Steroid Biochem. Mol. Biol, 64:187-191). It is thus possible that one of the responses of vascular smooth muscle cells to estrogen is the up-regulation of the G-protein γ-12 subunit mRNA resulting in an increase of G-protein γ-12 protein within the cell. A similar response may occur in vascular endothelial cells that also posses functional estrogen receptors. Given that the γ-12 subunit is the primary G-protein γ-subunit in (rat) smooth muscle cells, this increased level of γ-12 would be expected to have some, as yet undetermined, effect upon G-protein coupled signaling. This modulation of G-protein coupled signaling could play a role in the cardio-protective effects of estrogen through, for example, the modulation of the effects of various vasoactive molecules that signal through G protein coupled receptors.

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid encoding an estrogen-regulated GTP-binding protein gamma-12 subunit protein, wherein the protein comprises the amino acid sequence of SEQ ID NO: 1.

2. The nucleic acid of claim 1, wherein the nucleic acid is from a mouse.

3. The nucleic acid of claim 1, wherein the nucleic acid comprises a nucleotide sequence that is at least about 70% identical to SEQ ID NO: 2 or 3.

4. The nucleic acid of claim 1, wherein the nucleic acid comprises the nucleotide sequence of SEQ ID NO: 2 or 3.

5. An expression cassette comprising the nucleic acid of claim 1.

6. An isolated eukaryotic cell comprising the expression cassette of claim 5.

7. An isolated estrogen-regulated GTP-binding protein gamma-12 subunit protein, wherein the protein comprises the amino acid sequence of SEQ ID NO: 1.

8. The protein of claim 7, wherein the protein is a mouse protein.

9. An antibody that selectively binds to the estrogen-regulated GTP-binding protein gamma-12 subunit protein of claim 7, wherein the antibody does not bind to the estrogen-regulated GTP-binding protein gamma-12 subunit protein having the amino acid sequence of SEQ ID NO: 4.

10. A method of modulating estrogen signaling in a mammalian cell, the method comprising modulating the level of expression or activity of an estrogen-regulated GTP-binding protein gamma-12 subunit protein.

11. The method of claim 10, wherein said level of expression of said estrogen-regulated GTP-binding protein gamma-12 subunit protein is modulated by introducing a polynucleotide into said cell, whereby the presence or expression of said polynucleotide modulates said level of expression of said estrogen-regulated GTP-binding protein gamma-12 subunit protein.

12. The method of claim 11, wherein said polynucleotide encodes a full-length estrogen-regulated GTP-binding protein gamma-12 subunit protein, and wherein expression of said polynucleotide increases said level of expression of said estrogen-regulated GTP-binding protein gamma-12 subunit protein.

13. The method of claim 11, wherein said polynucleotide is an antisense sequence, and wherein the presence or expression of said polynucleotide decreases said level of expression of said estrogen-regulated GTP-binding protein gamma-12 subunit protein.

14. The method of claim 10, wherein a compound is administered to said cell, whereby said level of said expression or activity of said estrogen-regulated GTP-binding protein gamma-12 subunit is modulated.

15. The method of claim 10, wherein the estrogen signaling is mediated by an estrogen receptor alpha.

16. The method of claim 10, wherein the cell is present in a mammal.

17. The method of claim 16, wherein the cell is a vascular smooth muscle cell or a vascular endothelial cell.

18. The method of claim 16, wherein said level of expression or activity of said estrogen-regulated GTP-binding protein gamma-12 subunit protein is increased, whereby the development of atherosclerosis, osteoporosis, Alzheimer's disease or Parkinson's disease is inhibited in said mammal.

19. A method of detecting the presence of estrogen signaling in a mammalian cell, the method comprising detecting the expression of a nucleic acid encoding an estrogen-regulated GTP-binding protein gamma-12 subunit protein.

20. The method of claim 19, wherein said nucleic acid is the nucleic acid of claim 1.

21. The method of claim 19, wherein said presence of estrogen signaling in said cell is used in order to determine the responsiveness of said cell to estrogen.

22. The method of claim 19, wherein said presence of estrogen signaling in said cell is used in order to determine the tissue-specific distribution of estrogen signaling in a mammal.

23. The method of claim 19, wherein said expression of said nucleic acid in said cell is detected by detecting the expression or activity of an estrogen-regulated GTP-binding protein gamma-12 subunit protein.

24. The method of claim 19, wherein said protein is the protein of claim 7.

25. The method of claim 19, wherein said expression of said nucleic acid in said cell is detected by detecting the level of estrogen-regulated GTP-binding protein gamma-12 subunit mRNA in said cell.

26. The method of claim 19, wherein the estrogen signaling is mediated by an estrogen receptor alpha.

27. A method of identifying a compound capable of acting as an estrogen receptor agonist or antagonist, the method comprising:
(1) contacting a cell comprising an estrogen receptor with said compound; and
(2) determining the functional effect of said compound on said cell, wherein an increase in the level of estrogen-regulated GTP-binding protein gamma-12 subunit mRNA, protein or protein activity in said cell indicates that said compound is capable of acting as an estrogen receptor agonist, and wherein a decrease in the level of estrogen-regulated GTP-binding protein gamma-12 subunit mRNA, protein or protein activity in said cell indicates that said compound is capable of acting as an estrogen receptor antagonist.

28. The method of claim 27, wherein the estrogen receptor is an estrogen receptor alpha.

29. The method of claim 27, wherein said estrogen-regulated GTP-binding protein gamma-12 subunit mRNA has the sequence of SEQ ID NO: 1, or wherein said estrogen-regulated GTP-binding protein gamma-12 subunit protein comprises the amino acid sequence of SEQ ID No: 2 or 3.
